(19) United States
(12) Patent Application Publication
(10) Pub. No.: US 2010/0223682 A1
(43) Pub. Date: Sep. 2, 2010

(54) CASEIN AND METHODS OF USE THEREOF

(76) Inventors: Yitzhak Katz, Petach-Tikvah (IL); Michael Goldberg, Bet-Shemesh (IL)

Correspondence Address: Pearl Cohen Zedek Latzer, LLP, 1500 Broadway, 12th Floor, New York, NY, 10036, US

(21) Appl. No.: 12/649,489

(22) Filed: Dec. 30, 2009

Related U.S. Application Data
(60) Provisional application No. 61/141,554, filed on Dec. 30, 2008.

(51) Int. Cl. A01K 67/00; C07K 7/06; C07H 21/04; C12N 15/63; A61K 38/08; G01N 33/00

(52) U.S. Cl. 800/13; 530/328; 536/23.1; 435/320.1; 514/16; 436/86

(57) ABSTRACT

This invention provides an altered kosher kappa casein polypeptide having at least one altered amino acid in a defined amino acid stretch. The defined amino acid stretch contributes to an allergic reaction induced by a kosher wild-type casein polypeptide in a human subject. Moreover, the defined amino acid stretch is expressed solely in kosher animals. The invention further provides an expression system for expressing the altered kosher casein polypeptide.

### FIELD OF INVENTION

This invention provides an altered kosher kappa casein and methods of making and using the same.

### BACKGROUND OF THE INVENTION

Casein is the principal protein fraction of cows' milk. It accounts for about 80% of the protein content and is present in concentrations of 2.5-3.2%. Casein is a mixed complex of phosphoproteins existing in milk as colloidally dispersed micelles 50 to 600 nanometers in diameter. Caseins can be separated from the whey proteins of cows' milk by gel filtration, high-speed centrifugation, salting-out with appropriate concentrations of neutral salts, acid precipitation at pH 4.3-4.6, and coagulation with rennet (or other proteolytic enzymes), and as a coprecipitate with whey proteins. The first three methods yield preparations in essentially their native micellar state, but are impractical for commercial exploitation. Thus, commercial caseins are produced by methods more amenable to industrial practices. See also Micelle.

The early production of casein isolates is stimulated by their application in industrial products such as paper, glue, paint, and plastics. Thus the emphasis has shifted to their utilization in food systems, where they add enhanced nutritional and functional characteristics. They are widely used in the formulation of comminuted meat products, coffee whitener, processed cereal products, bakery products, and cheese analogs.

It was previously shown that children with Autism placed on a casein-free diet for eight weeks showed significant behavior improvements. In many cases, casein free diets are combined with gluten-free diets and are referred to as a gluten-free, casein-free diet.

In addition to being consumed in milk, casein is used in the manufacture of adhesives, binders, protective coatings, plastics (such as for knife handles and knitting needles), fabrics, food additives and many other products. It is commonly used by bodybuilders as a slow-digesting source of amino acids as opposed to the fast-digesting whey protein, and also as an extremely high source of glutamine (post-workout). Casein is frequently found in otherwise nondairy cheese substitutes to improve consistency, especially when melted.

One of the major allergeneic proteins in milk are the caseins. A phylogenetic comparison of beta-casein and kappa-casein genes demonstrates that the goat, sheep, cow, deer, and pronghorn casein genes are most closely related when compared to the milk casein of a camel, pig or zebra. “Kosher” animals are listed in the Old Testament as animals that the Jews were allowed to eat, and are generally described as animals which have split hooves (artiodactyls) and chew their cud (ruminants).

### SUMMARY OF THE INVENTION

This invention provides, an isolated altered kosher casein polypeptide, comprising: (1) at least one altered amino acid in the amino acid stretch encoded by SEQ ID No: 1 or SEQ ID No: 2 or (2) a substitution or a deletion in the position of Cys 31, Cys 32, or Cys 31 and Cys 32.

In another embodiment, the present invention provides a composition comprising an altered kosher casein polypeptide, wherein the casein polypeptide comprises: (1) at least one altered amino acid in the amino acid stretch encoded by SEQ ID No: 1 or SEQ ID No: 2; or (2) a substitution or a deletion in the position of Cys 31, Cys 32, or Cys 31 and Cys 32.

In another embodiment, the present invention provides a method of reducing the allergenicity of a kosher casein polypeptide, comprising the step of (1) altering at least one amino acid in the amino acid stretch encoded by SEQ ID No: 1 or SEQ ID No: 2; or (2) substituting or deleting the amino acid in the position of Cys 31, Cys 32, or both Cys 31 and Cys 32, thereby reducing the allergenicity of a casein polypeptide.

In another embodiment, the present invention provides an expression system comprising a first DNA sequence coding on expression for a kosher animal casein polypeptide comprising: (1) at least one altered amino acid in the amino acid stretch encoded by SEQ ID No: 1 or SEQ ID No: 2; or (2) a substitution or a deletion in the position of Cys 31, Cys 32, or Cys 31 and Cys 32.

In another embodiment, the present invention provides a transgenic kosher mammal, comprising a transgene encoding an altered kosher casein polypeptide which comprises (1) at least one altered amino acid in the amino acid stretch encoded by SEQ ID No: 1 or SEQ ID No: 2; or (2) a substitution or a deletion in the position of Cys 31, Cys 32, or Cys 31 and Cys 32.

### BRIEF DESCRIPTION OF THE FIGURES

FIG. 1 is a kappa casein amino acid sequence alignment scheme. The double sided arrow represents the amino acid stretch identified by SEQ ID No: 1 or SEQ ID No: 2.

FIG. 2 is a phylogenetic tree of kappa casein. The bar represents substitutions per 100 residues.

FIG. 3 is a micrograph of a gel demonstrating the expression of κ-casein in insect cell extracts. Twenty-five microliter of an in vitro translation reaction containing radiolabelled protein was loaded per lane and run on a 4-20 gradient Nu-Sep gel. The constructs in (A) were generated without any His₆ tags, while in (B) we demonstrate expressed bovine constructs generated with the signal sequence on the 5′ end and a His₆ at the 3′ end. Lucif=Luciferase positive control; Neg=Negative control (insect cell extract without any addition of mRNA); WT=Wildtype (native bovine κ-casein construct) Del=κ-casein with amino acids 104-111 deleted (Del_VPAKSCQA); Ile to Thr=Isoleucine at position 94 converted to threonine. Arrowheads denote the κ-casein construct.

### DETAILED DESCRIPTION OF THE INVENTION

This invention provides an isolated kosher casein polypeptide, comprising at least one altered amino acid in the amino acid stretch encoded by SEQ ID No: 1 or SEQ ID No: 2. In another embodiment, an isolated kosher casein polypeptide of the invention is an altered kosher casein polypeptide. In another embodiment, an isolated kosher casein polypeptide of the invention is an altered kosher kappa casein polypeptide.

In another embodiment, the invention provides an isolated kosher casein polypeptide, comprising a substitution or a deletion in the position of Cys 31, Cys 32, or Cys 31 and Cys 32.

In another embodiment, the invention provides an isolated kosher casein polypeptide, comprising (1) at least one altered amino acid in the amino acid stretch encoded by SEQ ID No: 1 or SEQ ID No: 2 and (2) a substitution or a deletion in the position of Cys 31, Cys 32, or Cys 31 and Cys 32.

In another embodiment, kosher animals are known to one of skill in the art. In another embodiment, a kosher animal is a mammals and a herbivore comprising a fully split hooves, who also chew their cud (ruminants). In another embodiment, a kosher animal is a bovine. In another embodiment, a kosher animal is a cow. In another embodiment, a kosher animal is a goat. In another embodiment, a kosher animal is a sheep. In another embodiment, a kosher animal is a deer. In another embodiment, a kosher animal is a buffalo. In another embodiment, a kosher animal is an antelope. In another embodiment, a kosher animal is a musk deer. In another embodiment, a kosher animal is a giraffe. In another embodiment, a kosher animal is a pronghorns.

In another embodiment, a kosher casein is a casein derived from a kosher animal. In another embodiment, a kosher casein is a casein comprising an amino acid sequence identical or homologous to a kosher animal casein. In another embodiment, a kosher casein comprises the 8 amino acids encoded by SEQ ID No: 1 or SEQ ID No: 2. In another embodiment, a kosher casein comprises a sequence homologous to SEQ ID No: 1 or SEQ ID No: 2. In another embodiment, a kosher casein comprises the amino acid sequence: VPAKSCQD (SEQ ID No: 1). In another embodiment, a kosher casein comprises the amino acid sequence: VPAKSCQA (SEQ ID No: 2).

In another embodiment, the term “altered amino acid” comprises a deleted amino acid. In another embodiment, the term “altered amino acid” comprises the substitution of an amino acid with another amino acid. In another embodiment, the term “altered amino acid” comprises the insertion of an amino acid.

In another embodiment, the term “amino acid stretch encoded by SEQ ID No: 1 or SEQ ID No: 2” refers to the region in the amino acid sequence of a kosher casein identified by SEQ ID No: 1 or SEQ ID No: 2. In another embodiment, the term “amino acid stretch encoded by SEQ ID No: 1 or SEQ ID No: 2” refers to the region in the amino acid sequence of a kosher kappa casein identified by SEQ ID No: 1 or SEQ ID No: 2.

“Modified kosher casein” or “altered kosher casein” refers, in another embodiment, to any kosher animal casein comprising at least a single mutation in SEQ ID No: 1 or SEQ ID No: 2 of a kosher casein amino acid sequence. “Modified kosher casein” or “altered kosher casein” refers, in another embodiment, to any kosher animal casein comprising at least a single mutation in DNA sequence encoding SEQ ID No: 1 or SEQ ID No: 2 of a DNA sequence encoding a kosher casein. “Modified kosher casein” or “altered kosher casein” refers, in another embodiment, to any kosher animal casein comprising a substitution or a deletion in the position of Cys 31, Cys 32, or Cys 31 and Cys 32. “Modified kosher casein” or “altered kosher casein” refers, in another embodiment, to any kosher animal casein comprising (1) at least one altered amino acid in the amino acid stretch encoded by SEQ ID No: 1 or SEQ ID No: 2; and (2) a substitution or a deletion in the position of Cys 31, Cys 32, or Cys 31 and Cys 32.

In another embodiment, the invention provides an altered kosher kappa casein polypeptide having at least one altered amino acid in a defined amino acid stretch. In another embodiment, the defined amino acid stretch is encoded by SEQ ID No: 1 or SEQ ID No: 2. In another embodiment, the defined amino acid stretch induces an allergic reaction induced by a wild-type kosher kappa casein polypeptide in a human subject. In another embodiment, a wild-type kosher kappa casein comprises a 3 dimensional structure that exposes certain epitopes that induce an allergic reaction in a human subject. In another embodiment, the defined amino acid stretch contributes to an allergic reaction induced by a wild-type kosher kappa casein polypeptide in a human subject. In another embodiment, an altered kosher kappa casein of the invention comprises a 3 dimensional structure characterized by dimensional structure characterized epitopes with a reduced ability to induce an allergic reaction in a subject. In another embodiment, an altered kosher kappa casein of the invention having at least one altered amino acid in a defined amino acid stretch encoded by SEQ ID No: 1 or SEQ ID No: 2 comprises a 3 dimensional structure characterized by reduced epitopes that induce an allergic reaction in a human subject. In another embodiment, an altered kosher kappa casein of the invention having at least one altered amino acid in a defined amino acid stretch encoded by SEQ ID No: 1 or SEQ ID No: 2 comprises a 3 dimensional structure that is less allergenic compared to the wild-type kosher kappa casein. In another embodiment, an altered kosher kappa casein of the invention having at least one altered amino acid in a defined amino acid stretch encoded by SEQ ID No: 1 or SEQ ID No: 2 is not allergenic.

In another embodiment, an altered kosher kappa casein polypeptide of the present invention comprises a substitution in the position of Cys 31. In another embodiment, an altered kosher kappa casein polypeptide of the present invention comprises a substitution in the position of Cys 32. In another embodiment, an altered kosher kappa casein polypeptide of the present invention comprises substitutions in Cys 31 and Cys 32. In another embodiment, the invention provides an altered kosher kappa casein polypeptide having at least one altered amino acid in a defined amino acid stretch as described hereinabove and a substitution in the position of Cys 31. In another embodiment, the invention provides an altered kosher kappa casein polypeptide having at least one altered amino acid in a defined amino acid stretch as described hereinabove and a substitution in the position of Cys 32. In another embodiment, the invention provides an altered kosher kappa casein polypeptide having at least one altered amino acid in a defined amino acid stretch as described hereinabove (SEQ ID No: 1 or SEQ ID No: 2) and a substitution or a deletion in the position of Cys 31, Cys 32, or both.

In another embodiment, modified kosher casein of the present invention is consumed by subjects diagnosed with casein allergy. In another embodiment, modified kosher casein of the present invention is consumed by subjects diagnosed with kappa casein allergy. In another embodiment, modified kosher casein of the present invention is consumed by subjects diagnosed with Cow milk allergy (CMA). In another embodiment, milk of the present invention is consumed by subjects diagnosed with Cow milk allergy (CMA). In another embodiment, the terms “modified kosher casein” and “altered kosher casein” are used interchangeably. In another embodiment, a “modified kosher casein” or an “altered kosher casein” comprises an amino acid sequence or encoded by a DNA sequence comprising SEQ ID Nos: 4, 5, 7, 8, 10, 11, 13, 14, and 16-21. In another embodiment, a “modified kosher casein” or an “altered kosher casein” consists an amino acid sequence or encoded by a DNA sequence comprising SEQ ID Nos: 4, 5, 7, 8, 10, 11, and 13-21.

In another embodiment, modified kosher casein of the present invention is consumed by human infants affected with IgE-mediated allergy to CMP. In another embodiment, modified kosher casein of the present invention is consumed by human infants affected with IgE-mediated allergy to goat derived milk. In another embodiment, modified kosher casein of the present invention is consumed by human infants affected with IgE-mediated allergy to sheep derived milk.

In another embodiment, IgE-mediated allergy to milk is assessed using the skin prick test (SPT). In another embodiment, IgE-mediated allergy to milk is assessed by measuring specific anti-casein IgE antibodies. In another embodiment, IgE-mediated allergy to milk is assessed by IgE-RAST. In another embodiment, IgE-mediated allergy to milk is assessed by an oral challenge test.

In another embodiment, IgE-mediated allergy to casein is diagnosed by a physician according to characteristic casein allergy signs and symptoms such as but not limited to: rash, vomiting, edema, shortness of breath and anaphylaxis. In another embodiment, theses signs and symptoms appear shortly after the ingestion of casein and/or cow, (goat, buffalo, sheep) milk. In another embodiment, theses signs and symptoms appear within 20 minutes after the ingestion of casein and/or cow, (goat, buffalo, sheep) milk. In another embodiment, theses signs and symptoms appear within 60 minutes hour after the ingestion of casein and/or cow, (goat, buffalo, sheep) milk. In another embodiment, theses signs and symptoms appear within 90 minutes after the ingestion of casein and/or cow, (goat, buffalo, sheep) milk. In another embodiment, theses signs and symptoms appear within 120 minutes after the ingestion of casein and/or cow, (goat, buffalo, sheep) milk. In another embodiment, theses signs and symptoms appear within 180 minutes after the ingestion of casein and/or cow, (goat, buffalo, sheep) milk. In another embodiment, theses signs and symptoms appear within 240 minutes after the ingestion of casein and/or cow, (goat, buffalo, sheep) milk.

In another embodiment, reduced allergenicity for the -kappa-casein would be reflected in a negative or reduced SPT. In another embodiment, reduced allergenicity for the -kappa-casein would be reflected in a decreased serum specific IgE to the -kappa-casein polypeptide. In another embodiment, reduced allergenicity for the -kappa-casein would be reflected in tolerance to the milk containing the -kappa-casein polypeptide in an oral challenge test.

In another embodiment, patients affected with atopic dermatitis and/or asthma can benefit from the compositions of the present invention comprising modified kosher casein.

In another embodiment, the modified kosher casein of the invention is a non-allergenic polypeptide. In another embodiment, the compositions of the invention are particularly intended for individuals at risk of milk protein allergy. In another embodiment, the use of non-allergenic peptides of milk proteins for the preparation of a hypoallergenic composition is intended for mammals susceptible to cow's milk allergy. In another embodiment, the use of non-allergenic peptides of milk proteins for the preparation of a hypoallergenic composition is intended for humans susceptible to cow's milk allergy.

In another embodiment, the present invention further provides a method of reducing the allergenicity of a kosher casein polypeptide, comprising the step of altering at least one amino acid of a kosher casein polypeptide in the amino acid stretch encoded by SEQ ID No: 1 or SEQ ID No: 2, thereby reducing the allergenicity of a casein polypeptide. In another embodiment, the present invention further provides a method of reducing the allergenicity of a kosher casein polypeptide, comprising the step of substituting or a deleting Cys 31, Cys 32, or both Cys 31 and Cys 32, thereby reducing the allergenicity of a casein polypeptide. In another embodiment, the present invention further provides a method of reducing the allergenicity of a kosher casein polypeptide, comprising the step of: (1) altering at least one amino acid of a kosher casein polypeptide in the amino acid stretch encoded by SEQ ID No: 1 or SEQ ID No: 2 and (2) substituting or deleting Cys 31, Cys 32, or both Cys 31 and Cys 32, thereby reducing the allergenicity of a casein polypeptide.

In another embodiment, the present invention further provides a method of reducing the allergenicity of a kosher casein polypeptide, comprising the step of deleting the 8 amino acids in SEQ ID No: 1 or SEQ ID No: 2 in a kosher casein polypeptide, thereby reducing the allergenicity of a casein polypeptide. In another embodiment, the present invention further provides a method of reducing the allergenicity of a kosher casein polypeptide, comprising the step of: (1) deleting the 8 amino acids in SEQ ID No: 1 or SEQ ID No: 2 in a kosher casein polypeptide and (2) substituting or deleting Cys 31, Cys 32, or both Cys 31 and Cys 32.

In another embodiment, the defined amino acid stretch is expressed preferentially in kosher animals. In another embodiment, the defined amino acid stretch is expressed solely in kosher animals. In another embodiment, the invention further provides an expression system for expressing the altered kosher casein polypeptide. In another embodiment, the invention further provides a genetically engineered animal comprising a transgene which encodes an altered kosher kappa casein of the invention.

In another embodiment, an altered kosher casein of the invention is a deletion mutant lacking 1 amino acid in any position within SEQ ID No: 1 or SEQ ID No: 2 of a kosher casein amino acid sequence. In another embodiment, an altered kosher casein of the invention is a deletion mutant lacking 2 amino acids in any 2 positions within SEQ ID No: 1 or SEQ ID No: 2 of a kosher casein amino acid sequence. In another embodiment, an altered kosher casein of the invention is a deletion mutant lacking 3 amino acids in any 3 positions within SEQ ID No: 1 or SEQ ID No: 2 of a kosher casein amino acid sequence. In another embodiment, an altered kosher casein of the invention is a deletion mutant lacking 4 amino acids in any 4 positions within SEQ ID No: 1 or SEQ ID No: 2 of a kosher casein amino acid sequence. In another embodiment, an altered kosher casein of the invention is a deletion mutant lacking 5 amino acids in any 5 positions within SEQ ID No: 1 or SEQ ID No: 2 of a kosher casein amino acid sequence. In another embodiment, an altered kosher casein of the invention is a deletion mutant lacking 6 amino acids in any 6 positions within SEQ ID No: 1 or SEQ ID No: 2 of a kosher casein amino acid sequence. In another embodiment, an altered kosher casein of the invention is a deletion mutant lacking 7 amino acids in any 7 positions within SEQ ID No: 1 or SEQ ID No: 2 of a kosher casein amino acid sequence. In another embodiment, an altered kosher casein of the invention is a deletion mutant lacking all 8 amino acids within SEQ ID No: 1 or SEQ ID No: 2 of a kosher casein amino acid sequence.

In another embodiment, an altered kosher casein of the invention is an insertion mutant comprising an additional 1 amino acid in any position within SEQ ID No: 1 or SEQ ID No: 2 of a kosher casein amino acid sequence. In another embodiment, an altered kosher casein of the invention is an insertion mutant comprising additional 2 amino acids in any position within SEQ ID No: 1 or SEQ ID No: 2 of a kosher casein amino acid sequence. In another embodiment, an altered kosher casein of the invention is an insertion mutant comprising additional 2 amino acids in any position within SEQ ID No: 1 or SEQ ID No: 3 of a kosher casein amino acid sequence. In another embodiment, an altered kosher casein of the invention is an insertion mutant comprising additional 4 amino acids in any position within SEQ ID No: 1 or SEQ ID No: 2 of a kosher casein amino acid sequence. In another embodiment, an altered kosher casein of the invention is an insertion mutant comprising additional 5-7 amino acids in any position within SEQ ID No: 1 or SEQ ID No: 2 of a kosher casein amino acid sequence. In another embodiment, an altered kosher casein of the invention is an insertion mutant comprising additional 7-10 amino acids in any position within SEQ ID No: 1 or SEQ ID No: 2 of a kosher casein amino acid sequence. In another embodiment, an altered kosher casein of the invention is an insertion mutant comprising additional 10-15 amino acids in any position within SEQ ID No: 1 or SEQ ID No: 2 of a kosher casein amino acid sequence. In another embodiment, an altered kosher casein of the invention is an insertion mutant comprising additional 15-30 amino acids in any position within SEQ ID No: 1 or SEQ ID No: 2 of a kosher casein amino acid sequence. In another embodiment, an altered kosher casein of the invention is an insertion mutant comprising additional 20-80 amino acids in any position within SEQ ID No: 1 or SEQ ID No: 2 of a kosher casein amino acid sequence.

In another embodiment, an altered kosher casein of the invention is a mutant comprising a substituted amino acid sequence in any position within SEQ ID No: 1 or SEQ ID No: 2 of a kosher casein amino acid sequence. In another embodiment, SEQ ID No: 1 or SEQ ID No: 2 is replaced with another sequence comprising 8 amino acids. In another embodiment, SEQ ID No: 1 or SEQ ID No: 2 is replaced with a sequence comprising 1-3 amino acids. In another embodiment, SEQ ID No: 1 or SEQ ID No: 2 is replaced with a sequence comprising 2-6 amino acids. In another embodiment, SEQ ID No: 1 or SEQ ID No: 2 is replaced with a sequence comprising 3-8 amino acids. In another embodiment, SEQ ID No: 1 or SEQ ID No: 2 is replaced with a sequence comprising 5-10 amino acids. In another embodiment, SEQ ID No: 1 or SEQ ID No: 2 is replaced with a sequence comprising 10-30 amino acids. In another embodiment, SEQ ID No: 1 or SEQ ID No: 2 is replaced with a sequence comprising 20-60 amino acids.

In another embodiment, the isolated kosher casein of the present invention is a modified kosher casein. In another embodiment, the isolated kosher casein of the present invention is modified in along SEQ ID No: 1 or SEQ ID No: 2. In another embodiment, the isolated modified kosher casein of the present invention comprises the amino acid sequence of a kosher casein wherein SEQ ID No: 1 or SEQ ID No: 2 are excluded. In another embodiment, the modification of the kosher casein of the present invention comprises the deletion of the amino acid sequence set fourth in SEQ ID No: 1 or SEQ ID No: 2. In another embodiment, the modification of the kosher casein of the present invention comprises the deletion of at least one amino acid from the amino acid sequence set fourth in SEQ ID No: 1 or SEQ ID No: 2. In another embodiment, the modification of a kosher casein of the present invention comprises at least a single mutation in the DNA sequence encoding the amino acid sequence set fourth in SEQ ID No: 1 or SEQ ID No: 2 which results in the substitution of at least one amino acid with at least another amino acid. In another embodiment, the modification of a kosher casein of the present invention comprises at least a single amino acid insertion in the amino acid sequence set fourth in SEQ ID No: 1 or SEQ ID No: 2. In another embodiment, the modification of a kosher casein of the present invention comprises at least a single amino acid deletion in the amino acid sequence set fourth in SEQ ID No: 1 or SEQ ID No: 2. In another embodiment, the invention provides that an altered kosher kappa casein polypeptide comprises: (a) at least one altered amino acid in a defined amino acid stretch encoded by SEQ ID No: 1 or SEQ ID No: 2, or (b) a substitution or a deletion in the position of Cys 31, Cys 32, or both Cys 31 and Cys 32; or (c) at least one altered amino acid in a defined amino acid stretch encoded by SEQ ID No: 1 or SEQ ID No: 2 and a substitution or a deletion in the position of Cys 31, Cys 32, or both Cys 31 and Cys 32. In another embodiment, the terms modified and altered are used interchangeably.

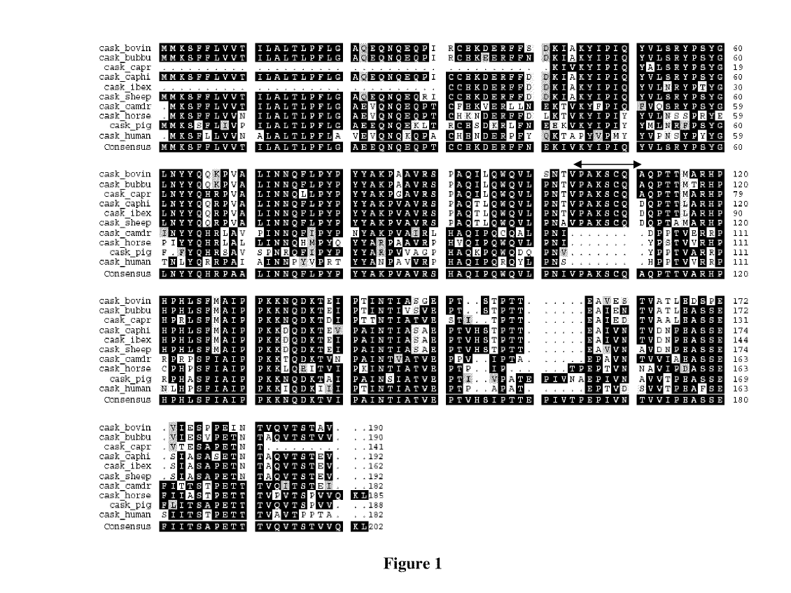

In another embodiment, SEQ ID No: 1 or SEQ ID No: 2 are located in the middle of the casein polypeptide. In another embodiment, SEQ ID No: 1 or SEQ ID No: 2 are located in between amino acid number 50 to amino acid number 120, inclusive, of the kosher casein amino acid sequence. In another embodiment, SEQ ID No: 1 or SEQ ID No: 2 are located in between amino acid number 60 to amino acid number 120, inclusive, of the kosher casein amino acid sequence. In another embodiment, SEQ ID No: 1 or SEQ ID No: 2 are located in between amino acid number 104 to amino acid number 111, inclusive, of the kosher casein amino acid sequence. In another embodiment, SEQ ID No: 2 is located in between amino acid number 104 to amino acid number 111, inclusive, of a bovine casein amino acid sequence (see FIG. 1). In another embodiment, SEQ ID No: 2 is located in between amino acid number 104 to amino acid number 111, inclusive, of a buffalo casein amino acid sequence (see FIG. 1). In another embodiment, SEQ ID No: 1 is located in between amino acid number 104 to amino acid number 111, inclusive, of a Caphi (goat) casein amino acid sequence (see FIG. 1). In another embodiment, SEQ ID No: 1 is located in between amino acid number 104 to amino acid number 111, inclusive, of a sheep casein amino acid sequence (see FIG. 1). In another embodiment, SEQ ID No: 2 is located in between amino acid number 63 to amino acid number 70, inclusive, of Roe Deer (Capreolus) casein amino acid sequence (see FIG. 1). In another embodiment, SEQ ID No: 1 is located in between amino acid number 74 to amino acid number 81, inclusive, of IBEX casein amino acid sequence (see FIG. 1).

In another embodiment, the present invention provides that the altered kosher casein of the present invention is characterized by reduced allergenicity when consumed by a human subject allergic to wild-type kosher casein. In another embodiment, the present invention provides that the altered kosher casein of the present invention is characterized by reduced allergenicity when contacted with a human subject allergic to wild-type kosher casein. In another embodiment, the present invention provides that the presence of SEQ ID No: 1 or SEQ ID No: 2 in the amino acid sequence of a kosher casein induces an allergic reaction in a human subject. In another embodiment, the present invention provides that the presence of SEQ ID No: 1 or SEQ ID No: 2 in the amino acid sequence of a kosher casein induces an allergic reaction in a human subject consuming a kosher casein. In another embodiment, the altered kosher casein of the present invention is characterized by reduced allergenicity. In another embodiment, the altered kosher casein of the present invention is non-allergenic. In another embodiment, altering or deleting SEQ ID No: 1 or SEQ ID No: 2 in the amino acid sequence encoding a kosher casein results in a kosher casein with reduced allergenicity. In another embodiment, altering or deleting SEQ ID No: 1 or SEQ ID No: 2 in the amino acid sequence encoding a kosher casein results in non-allergenic kosher casein.

In another embodiment, the present invention provides that the presence Cys 31, Cys 32, or both Cys 31 and Cys 32 in the amino acid sequence of a kosher casein induces an allergic reaction in a human subject. In another embodiment, the present invention provides that the presence of : (1) SEQ ID No: 1 or SEQ ID No: 2 and (2) Cys 31, Cys 32, or both Cys 31 and Cys 32in the amino acid sequence of a kosher casein induces an allergic reaction in a human subject consuming a kosher casein. In another embodiment, substituting or deleting Cys 31, Cys 32, or both Cys 31 and Cys 32 in the amino acid sequence encoding a kosher casein results in a kosher casein with reduced allergenicity. In another embodiment, substituting or deleting Cys 31, Cys 32, or both Cys 31 and Cys 32 in the amino acid sequence encoding a kosher casein results in non-allergenic kosher casein.

In another embodiment, domestic cow (Bos taurus) casein kappa protein has amino acids the sequence:
MMKSFFLVVTILALTLPFLGAQEQNQEQPIRCEKDERFFSDKIAKYIPIQYVLSRYPSYGLNY YQQKPVALINNQFLPYPYYAKPAAVRSPAQILQWQVLSNTVPAKSCQAQPTTMARHPHPHL SFMAIPPKKNQDKTEIPTINTIASGEPTSTPTTEAVESTVATLEDSPEVIESPPEINTVQVTSTA
   V (SEQ ID No: 3). In another embodiment, domestic cow casein is homologous to the sequence set forth in SEQ ID No: 3. Each possibility represents a separate embodiment of the present invention.

In another embodiment, the domestic cow amino acids sequence is set forth in GenBank Accession number NP_{—}776719. In another embodiment, the domestic cow mRNA sequence is set forth in GenBank Accession number NM_{—}174294. Each possibility represents a separate embodiment of the present invention.

In another embodiment, the altered kosher casein of the present invention is characterized by altering at least one underlined amino acid (SEQ ID No: 2) in SEQ ID No: 3. In another embodiment, the altered kosher casein of the present invention is characterized by the deletion of at least one underlined amino acid (SEQ ID No: 2) in SEQ ID No: 3. In another embodiment, the altered kosher casein of the present invention is characterized by the deletion of the underlined amino acids (SEQ ID No: 2) in SEQ ID No: 3. In another embodiment, the altered kosher casein of the present invention is characterized by a substitution in the double underlined cysteine (Cys 32) residue in SEQ ID No: 3. In another embodiment, the altered kosher casein of the present invention is characterized by a substitution in the position of Cys 32 and by the deletion of at least one underlined amino acid (SEQ ID No: 2) in SEQ ID No: 3. In another embodiment, the altered kosher casein of the present invention is characterized by a substitution in the position of Cys 32 and the deletion of the underlined amino acids (SEQ ID No: 2) in SEQ ID No: 3.

In another embodiment, an altered kosher casein kappa protein has the amino acids sequence:
MMKSFFLVVTILALTLPFLGAQEQNQEQPIRCEKDERFFSDKIAKYIPIQYVLSRYPSYGLNY YQQKPVALINNQFLPYPYYAKPAAVRSPAQILQWQVLSNTQPTTMARHPHPHLSFMAIPPK KNQDKTEIPTINTIASGEPTSTPTTEAVESTVATLEDS PEVIESPPEINTVQVTSTAV (SEQ ID No: 4). In another embodiment, an altered kosher casein kappa protein of the present invention is homologous to the sequence set forth in SEQ ID No: 4. In another embodiment, an altered kosher casein kappa protein of the present invention further comprises a substitution in the position of Cys 32 (double underlined) of SEQ ID No: 4. Each possibility represents a separate embodiment of the present invention.

In another embodiment, an altered kosher casein kappa protein has the amino acids sequence:
MMKSFFLVVTILALTLPFLGAQEQNQEQPIRCEKDERFFSDKIAKYIPIQYVLSRYPSYGLNY YQQKPVALINNQFLPYPYYAKPAAVRSPAQILQWQVLSNTAQPTTMARHPHPHLSFMAIPP KKNQDKTEIPTINTIASGEPTSTPTTEAVESTVATLEDSPEVIESPPEINTVQVTSTAV (SEQ ID No: 5). In another embodiment, an altered kosher casein kappa protein of the present invention is homologous to the sequence set forth in SEQ ID No: 5. In another embodiment, an altered kosher casein kappa protein of the present invention further comprises a substitution in the position of Cys 32 (double underlined) of SEQ ID No: 5. Each possibility represents a separate embodiment of the present invention.

In another embodiment, water buffalo (Bubalus bubalis) casein kappa protein has amino the sequence:
MMKSFFLVVTILALTLPFLGAQEQNQEQPIRCEKEERFFNDKIAKYIPIQYVLSRYPSYGLNY YQQKPVALINNQFLPYPYYAKPAAVRSPAQILQWQVLPNTVPAKSCQAQPTTMTRHPHPHL SFMAIPPKKNQDKTEIPTINTIVSVEPTSTPITEAIENTVATLEASSEVIESVPETNTAQVTST
   (SEQ ID No: 6). In another embodiment, buffalo casein is homologous to the sequence set forth in SEQ ID No: 6. Each possibility represents a separate embodiment of the present invention.

In another embodiment, the buffalo amino acids sequence is set forth in GenBank Accession number NP_{—}776719. In another embodiment, the buffalo mRNA sequence is set forth in GenBank Accession number AY750857. Each possibility represents a separate embodiment of the present invention.

In another embodiment, the altered kosher casein of the present invention is characterized by altering at least one underlined amino acid (SEQ ID No: 2) in SEQ ID No: 6. In another embodiment, the altered kosher casein of the present invention is characterized the deletion of at least one underlined amino acid (SEQ ID No: 2) in SEQ ID No: 6. In another embodiment, the altered kosher casein of the present invention is characterized by the deletion of the underlined amino acids (SEQ ID No: 2) in SEQ ID No: 6. In another embodiment, the altered kosher casein of the present invention is characterized by a substitution in the double underlined cysteine (Cys 32) residue in SEQ ID No: 6. In another embodiment, the altered kosher casein of the present invention is characterized by a substitution in the position of Cys 32 and by the deletion of at least one underlined amino acid (SEQ ID No: 2) in SEQ ID No: 6. In another embodiment, the altered kosher casein of the present invention is characterized by a substitution in the position of Cys 32 and the deletion of the underlined amino acids (SEQ ID No: 2) in SEQ ID No: 6.

In another embodiment, an altered kosher casein kappa protein has the amino acids sequence:
MMKSFFLVVTILALTLPFLGAQEQNQEQPIRCEKEERFFNDKIAKYIPIQYVLSRYPSYGLNY YQQKPVALINNQFLPYPYYAKPAAVRSPAQILQWQVLPNTQPTTMTRHPHPHLSFMAIPPK KNQDKTEIPTINTIVSVEPTSTPITEAIENTVATLEASSEVIESVPETNTAQVTST (SEQ ID No: 7). In another embodiment, an altered kosher casein kappa protein of the present invention is homologous to the sequence set forth in SEQ ID No: 7. In another embodiment, an altered kosher casein kappa protein of the present invention further comprises a substitution in the position of Cys 32 (double underlined) of SEQ ID No: 7. Each possibility represents a separate embodiment of the present invention.

In another embodiment, an altered kosher casein kappa protein has the amino acids sequence:
MMKSFFLVVTILALTLPFLGAQEQNQEQPIRCEKEERFFNDKIAKYIPIQYVLSRYPSYGLNY YQQKPVALINNQFLPYPYYAKPAAVRSPAQILQWQVLPNTAQPTTMTRHPHPHLSFMAIPP KKNQDKTEIPTINTIVSVEPTSTPITEAIENTVATLEASSEVIESVPETNTAQVTST (SEQ ID No: 8). In another embodiment, an altered kosher casein kappa protein of the present invention is homologous to the sequence set forth in SEQ ID No: 8. In another embodiment, an altered kosher casein kappa protein of the present invention further comprises a substitution in the position of Cys 32 (double underlined) of SEQ ID No: 8. Each possibility represents a separate embodiment of the present invention.

In another embodiment, goat (Capra hircus) casein kappa protein has the amino acids sequence:
MMKSFFLVVTILALTLPFLGAQEQNQEQPICCEKDERFFDDKIAKYIPIQYVLSRYPSYGLNY YQQRPVALINNQFLPYPYYAKPVAVRSPAQTLQWQVLPNTVPAKSCQDQPTTLARHPHPHL SFMAIPPKKDQDKTEVPAINTIASAEPTVHSTPTTEAIVNTVDNPEASSESIASASETNTAQVT STEV (SEQ ID No: 9). In another embodiment, goat casein is homologous to the sequence set forth in SEQ ID No: 9. Each possibility represents a separate embodiment of the present invention. In another embodiment, the goat amino acids sequence is set forth in GenBank Accession number P02670. Each possibility represents a separate embodiment of the present invention.

In another embodiment, the altered kosher casein of the present invention is characterized by altering at least one underlined amino acid (SEQ ID No: 1) in SEQ ID No: 9. In another embodiment, the altered kosher casein of the present invention is characterized by deleting at least one underlined amino acid (SEQ ID No: 1) in SEQ ID No: 9. In another embodiment, the altered kosher casein of the present invention is characterized by the deletion of the underlined amino acids (SEQ ID No: 1) in SEQ ID No: 9. In another embodiment, the altered kosher casein of the present invention is characterized by a substitution in at least on of the double underlined cysteine (Cys 31 and Cys 32) residue in SEQ ID No: 9. In another embodiment, the altered kosher casein of the present invention is characterized by a substitution in the position of Cys 31 and by the deletion of at least one underlined amino acid (SEQ ID No: 2) in SEQ ID No: 9. In another embodiment, the altered kosher casein of the present invention is characterized by a substitution in the position of Cys 32 and by the deletion of at least one underlined amino acid (SEQ ID No: 2) in SEQ ID No: 9. In another embodiment, the altered kosher casein of the present invention is characterized by substitutions in Cys 31 and Cys 32 and by the deletion of at least one underlined amino acid (SEQ ID No: 2) in SEQ ID No: 9. In another embodiment, the altered kosher casein of the present invention is characterized by a substitution in the position of Cys 31 and the deletion of the underlined amino acids (SEQ ID No: 2) in SEQ ID No: 9. In another embodiment, the altered kosher casein of the present invention is characterized by a substitution in the position of Cys 32 and the deletion of the underlined amino acids (SEQ ID No: 2) in SEQ ID No: 9. In another embodiment, the altered kosher casein of the present invention is characterized by substitutions in Cys 31 and Cys 32 and the deletion of the underlined amino acids (SEQ ID No: 2) in SEQ ID No: 9.

In another embodiment, an altered kosher casein kappa protein has the amino acids sequence:
MMKSFFLVVTILALTLPFLGAQEQNQEQPICCEKDERFFDDKIAKYIPQYVLSRYPSYGLNY YQQRPVALINNQFLPYPYYAKPVAVRSPAQTLQWQVLPNTQPTTLARHPHPHLSFMAIPPK KDQDKIEVPAINTIASAEPTVHSTPTTTEAIVNTVDNPEASSESIASASETNTAQVTSTEV (SEQ ID No: 10). In another embodiment, an altered kosher casein kappa protein of the present invention is homologous to the sequence set forth in SEQ ID No: 10. In another embodiment, the altered kosher casein of the present invention is characterized by substitutions in Cys 31 and Cys 32 (double underlined) of SEQ ID No: 10. In another embodiment, the altered kosher casein of the present invention is characterized by a substitution in the position of Cys 31 of SEQ ID No: 10. In another embodiment, the altered kosher casein of the present invention is characterized by a substitution in the position of Cys 32 of SEQ ID No: 10. Each possibility represents a separate embodiment of the present invention.

In another embodiment, an altered kosher casein kappa protein has the amino acids sequence:
MMKSFFLVVTILALTLPFLGAQEQMQEQPICCEKDERFFDDKIAKYIPIQYVLSRYPSYGLNY YQQRPVALINNQFLPYPYYAKPVAVRSPAQTLQWQVLPNTDQPTTLARHPHPHLSFMAIPP KKDQDKTEVPAINTIASAEPTVHSTPTTEAIVNTVDNPEASSESIASASETNTAQVTSTEV
   (SEQ ID No: 11). In another embodiment, an altered kosher casein kappa protein of the present invention is homologous to the sequence set forth in SEQ ID No: 11. In another embodiment, the altered kosher casein of the present invention is characterized by substitutions in Cys 31 and Cys 32 (double underlined) of SEQ ID No: 11. In another embodiment, the altered kosher casein of the present invention is characterized by a substitution in the position of Cys 31 of SEQ ID No: 11. In another embodiment, the altered kosher casein of the present invention is characterized by a substitution in the position of Cys 32 of SEQ ID No: 11. Each possibility represents a separate embodiment of the present invention.

In another embodiment, sheep (Ovis aries) casein kappa protein has the amino acids sequence:
MMKSFFLVVTILALTLPFLGAQEQNQEQRICCEKDERFFDDKIAKYIPIQYVLSRYPSYGLNY YQQRPVALINNQFLPYPYYAKPVAVRSPAQTLQWQVLPNAVPAKSCQDQPTAMARHPHPH LSFMAIPPKKDQDKTEIPAINTIASAEPTVHSTPTTEAVVNAVDNPEASSESIASAPETNTAQV TSTEV (SEQ ID No: 12). In another embodiment, sheep casein is homologous to the sequence set forth in SEQ ID No: 12. In another embodiment, the sheep casein sequence is set forth in GenBank Accession number P02669. In another embodiment, the sheep casein is homologous to the sequence set forth in GenBank Accession number P02669. Each possibility represents a separate embodiment of the present invention.

In another embodiment, the altered kosher casein of the present invention is characterized by altering at least one underlined amino acid (SEQ ID No: 1) in SEQ ID No: 12. In another embodiment, the altered kosher casein of the present invention is characterized by deleting at least one underlined amino acid (SEQ ID No: 1) in SEQ ID No: 12. In another embodiment, the altered kosher casein of the present invention is characterized by the deletion of the underlined amino acids (SEQ ID No: 1) in SEQ ID No: 12. In another embodiment, the altered kosher casein of the present invention is characterized by substitutions in Cys 31 and Cys 32 (double underlined) and by the deletion of at least one underlined amino acid (SEQ ID No: 1) in SEQ ID No: 12. In another embodiment, the altered kosher casein of the present invention is characterized by a substitution in the position of Cys 31 and the deletion of the underlined amino acids (SEQ ID No: 1) in SEQ ID No: 12. In another embodiment, the altered kosher casein of the present invention is characterized by a substitution in the position of Cys 32 and the deletion of the underlined amino acids (SEQ ID No: 1) in SEQ ID No: 12. In another embodiment, the altered kosher casein of the present invention is characterized by substitutions in Cys 31 and Cys 32 and the deletion of the underlined amino acids (SEQ ID No: 1) in SEQ ID No: 12.

In another embodiment, an altered kosher casein kappa protein has the amino acids sequence:
MMKSFFLVVTILALTLPFLGAQEQNQEQRICCEKDERFFDDKIAKYIPIQYVLSRYPSYGLNY YQQRPVALINNQFLPYPYYAKPVAVRSPAQTLQWQVLPNAQPTAMARHPHPHLSFMAIPPK KDQDKTEIPAINTIASAEPTVHSTPTTEAVVNAVDNPEASSESIASAPETNTAQVTSTEV (SEQ ID No: 13). In another embodiment, an altered kosher casein kappa protein of the present invention is homologous to the sequence set forth in SEQ ID No: 13. In another embodiment, the altered kosher casein of the present invention is characterized by substitutions in Cys 31 and Cys 32 (double underlined) of SEQ ID No: 13. In another embodiment, the altered kosher casein of the present invention is characterized by a substitution in the position of Cys 31 of SEQ ID No: 13. In another embodiment, the altered kosher casein of the present invention is characterized by a substitution in the position of Cys 32 of SEQ ID No: 13. Each possibility represents a separate embodiment of the present invention. Each possibility represents a separate embodiment of the present invention.

In another embodiment, an altered kosher casein kappa protein has the amino acids sequence:
MMKSFFLVVTILALTLPFLGAQEQNQEQRICCEKDERFFDDKIAKYIPIQYVLSRYPSYGLNY YQQRPVALINNQFLPYPYYAKPVAVRSPAQTLQWQVLPNADQPTAMARHPHPHLSFMAIPP KKDQDKTEIPAINTIASAEPTVHSTPTTEAVVNAVDNPEASSESIASAPETNTAQVTSTEV
   (SEQ ID No: 14). In another embodiment, an altered kosher casein kappa protein of the present invention is homologous to the sequence set forth in SEQ ID No: 14. In another embodiment, the altered kosher casein of the present invention is characterized by substitutions in Cys 31 and Cys 32 (double underlined) of SEQ ID No: 14. In another embodiment, the altered kosher casein of the present invention is characterized by a substitution in the position of Cys 31 of SEQ ID No: 14. In another embodiment, the altered kosher casein of the present invention is characterized by a substitution in the position of Cys 32 of SEQ ID No: 14. Each possibility represents a separate embodiment of the present invention. Each possibility represents a separate embodiment of the present invention.

Homology is, in another embodiment, is determined by computer algorithm for sequence alignment, by methods well described in the art. For example, computer algorithm analysis of nucleic acid sequence homology can include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to SEQ ID No: 1-14 of greater than 70%. In another embodiment, “homology” refers to identity to SEQ ID No: 1-14 of greater than 72%. In another embodiment, “homology” refers to identity to SEQ ID No: 1-14 of greater than 75%. In another embodiment, “homology” refers to identity to SEQ ID No: 1-14 of greater than 78%. In another embodiment, “homology” refers to identity to SEQ ID No: 1-14 of greater than 80%. In another embodiment, “homology” refers to identity to SEQ ID No: 1-14 of greater than 82%. In another embodiment, “homology” refers to identity to SEQ ID No: 1-14 of greater than 83%. In another embodiment, “homology” refers to identity to SEQ ID No: 1-14 of greater than 85%. In another embodiment, “homology” refers to identity to SEQ ID No: 1 of greater than 87%. In another embodiment, “homology” refers to identity to SEQ ID No: 1-14 of greater than 88%. In another embodiment, “homology” refers to identity to SEQ ID No: 1-14 of greater than 90%. In another embodiment, “homology” refers to identity to SEQ ID No: 1-14 of greater than 92%. In another embodiment, “homology” refers to identity to SEQ ID No: 1-14 of greater than 93%. In another embodiment, “homology” refers to identity to SEQ ID No: 1-14 of greater than 95%. In another embodiment, “homology” refers to identity to SEQ ID No: 1-14 of greater than 96%. In another embodiment, “homology” refers to identity to SEQ ID No: 1-14 of greater than 97%. In another embodiment, “homology” refers to identity to SEQ ID No: 1-14 of greater than 98%. In another embodiment, “homology” refers to identity to SEQ ID No: 1-14 of greater than 99%. In another embodiment, “homology” refers to identity to SEQ ID No: 1-14 of 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.). In other embodiments, methods of hybridization are carried out under moderate to stringent conditions, to the complement of a DNA encoding a native casein polypeptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

In another embodiment of the present invention, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA is, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy amino acids et al, Genes & Devel 16:2491-96 and references cited therein). DNA can be, in other embodiments, in form of plasmid DNA, viral DNA, linear DNA, or chromosomal DNA or derivatives of these groups. In addition, these forms of DNA and RNA can be single, double, triple, or quadruple stranded. The term also includes, in another embodiment, artificial nucleic acids that contain other types of backbones but the same bases. In one embodiment, the artificial nucleic acid is a PNA (polypeptide nucleic acid). PNA contain polypeptide backbones and nucleotide bases and are able to bind, in one embodiment, to both DNA and RNA molecules. In another embodiment, the nucleotide is oxetane modified. In another embodiment, the nucleotide is modified by substitution of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or polypeptide homology for any amino acids sequence listed herein is determined, in another embodiment, by methods well described in the art, including immunoblot analysis, or via computer algorithm analysis of amino acids sequences, utilizing any of a number of software packages available, via established methods. Some of these packages include the FASTA, BLAST, MPsrch or Scanps packages, and, in another embodiment, employ the use of the Smith and Waterman algorithms, and/or global/local or BLOCKS alignments for analysis, for example. Each method of determining homology represents a separate embodiment of the present invention.

In another embodiment, the isolated altered kosher casein of the present invention is produced in a bacterial cell. In another embodiment, altered kosher casein comprising SEQ ID No: 4, 5, 7, 8, 10, 11, 13 or, 14 is produced in a bacterial cell. In another embodiment, altered kosher casein comprising a substitution or a deletion in the position of Cys 31, Cys 32, or Cys 31 and Cys 32is produced in a bacterial cell. In another embodiment, altered kosher kappa casein of the present invention comprising: (1) SEQ ID No: 4, 5, 7, 8, 10, 11, 13 or, 14 and (2) a substitution or a deletion in the position of Cys 31, Cys 32, or Cys 31 and Cys 32 is produced in a bacterial cell. In another embodiment, methods of inserting a cDNA sequence of an altered kosher casein of the present invention into a bacterial vector are known to one of skill in the art. In another embodiment, bacterial strains that can be used to produce the altered kosher casein of the present invention are known to one of skill in the art. In another embodiment, the bacterially produced altered kosher casein of the present invention is further tagged. In another embodiment, tagging improves isolation of a kosher casein of the present invention. In another embodiment, bacterially produced isolated altered kosher casein of the present invention is of high purity. In another embodiment, high purity altered kosher casein is above 80% pure. In another embodiment, high purity altered kosher casein is above 85% pure. In another embodiment, high purity altered kosher casein is above 90% pure. In another embodiment, high purity altered kosher casein is above 95% pure. In another embodiment, high purity altered kosher casein is above 98% pure. In another embodiment, high purity altered kosher casein is above 99% pure.

In another embodiment, the isolated altered kosher casein of the present invention is produced in a prokaryotic cell. In another embodiment, the isolated altered kosher casein of the present invention is produced in a yeast cell. In another embodiment, altered kosher casein comprising SEQ ID No: 4, 5, 7, 8, 10, 11, 13 or, 14 is produced in a yeast cell. In another embodiment, altered kosher casein comprising a substitution or a deletion in the position of Cys 31, Cys 32, or Cys 31 and Cys 32is produced in a yeast cell. In another embodiment, altered kosher kappa casein of the present invention comprising: (1) SEQ ID No: 4, 5, 7, 8, 10, 11, 13 or, 14 and (2) a substitution or a deletion in the position of Cys 31, Cys 32, or Cys 31 and Cys 32 is produced in a yeast cell. In another embodiment, the isolated altered kosher casein of the present invention is produced in an insect cell. In another embodiment, the isolated altered kosher casein of the present invention is produced in a cell extract. In another embodiment, the isolated altered kosher casein of the present invention is produced in an insect cell extract.

In another embodiment, methods of inserting a DNA or a cDNA sequence of an altered kosher casein of the present invention into a yeast vector are known to one of skill in the art. In another embodiment, yeast strains that can be used to produce the altered kosher casein of the present invention are known to one of skill in the art. In another embodiment, yeast produced altered kosher casein of the present invention is further tagged. In another embodiment, tagging improves isolation of a kosher casein of the present invention. In another embodiment, yeast produced isolated altered kosher casein of the present invention is of high purity. In another embodiment, high purity altered kosher casein is above 80% pure. In another embodiment, high purity altered kosher casein is above 85% pure. In another embodiment, high purity altered kosher casein is above 90% pure. In another embodiment, high purity altered kosher casein is above 95% pure. In another embodiment, high purity altered kosher casein is above 98% pure. In another embodiment, high purity altered kosher casein is above 99% pure.

In another embodiment, the isolated altered kosher casein of the present invention is produced in a mammalian cell. In another embodiment, altered kosher casein comprising SEQ ID No: 4, 5, 7, 8, 10, 11, 13 or, 14 is produced in a mammalian cell. In another embodiment, altered kosher casein comprising a substitution or a deletion in the position of Cys 31, Cys 32, or Cys 31 and Cys 32 is produced in a mammalian cell. In another embodiment, the isolated altered kosher casein of the present invention is produced in a mammalian cell. In another embodiment, altered kosher kappa casein of the present invention comprising: (1) SEQ ID No: 4, 5, 7, 8, 10, 11, 13 or, 14 and (2) a substitution or a deletion in the position of Cys 31, Cys 32, or Cys 31 and Cys 32 is produced in a mammalian cell. In another embodiment, the mammalian cell is a cell derived from a cell line. In another embodiment, the mammalian cell is a cell within a tissue. In another embodiment, the mammalian cell is a primary cell culture cell. In another embodiment, methods of inserting a DNA or a cDNA sequence of an altered kosher casein of the present invention into a mammalian vector are known to one of skill in the art. In another embodiment, methods of inserting the vector and or transfection will be adapted according to the cell type that is being used as the producer of the altered kosher casein of the present invention. In another embodiment, mammalian produced altered kosher casein of the present invention is further tagged. In another embodiment, tagging improves isolation of a kosher casein of the present invention. In another embodiment, mammalian produced isolated altered kosher casein of the present invention is of high purity. In another embodiment, high purity altered kosher casein is above 80% pure. In another embodiment, high purity altered kosher casein is above 85% pure. In another embodiment, high purity altered kosher casein is above 90% pure. In another embodiment, high purity altered kosher casein is above 95% pure. In another embodiment, high purity altered kosher casein is above 98% pure. In another embodiment, high purity altered kosher casein is above 99% pure.

In another embodiment, the isolated altered kosher casein of the present invention is produced in a breast cell. In another embodiment, the isolated altered kosher casein of the present invention is produced in a breast alveoli cell.

In another embodiment, the isolated altered kosher casein of the present invention is produced in a breast alveoli cell of a kosher animal. In another embodiment, the isolated altered kosher casein of the present invention is produced in a breast alveoli cell of a lab animal. In another embodiment, the isolated altered kosher casein of the present invention is produced in a breast alveoli cell of a transgenic kosher animal. In another embodiment, methods of creating a transgenic kosher animal that produces the altered kosher casein of the present invention in its breast alveoli cells are known to one of skill in the art. In another embodiment, the isolated altered kosher casein of the present invention is a constituent of milk. In another embodiment, altered kosher casein of the invention is produced in a breast alveoli cell of a kosher animal. In another embodiment, altered kosher casein comprising SEQ ID No: 4, 5, 7, 8, 10, 11, 13 or, 14 is produced in a breast alveoli cell of a kosher animal. In another embodiment, altered kosher casein comprising a substitution or a deletion in the position of Cys 31, Cys 32, or Cys 31 and Cys is produced in a breast alveoli cell of a kosher animal. In another embodiment, altered kosher kappa casein of the present invention comprising: (1) SEQ ID No: 4, 5, 7, 8, 10, 11, 13 or, 14 and (2) a substitution or a deletion in the position of Cys 31, Cys 32, or Cys 31 and Cys 32 is produced in a breast alveoli cell of a kosher animal.

In another embodiment, the present invention provides an expression system comprising a first DNA sequence coding on expression for a kosher animal casein polypeptide comprising (1) at least one altered amino acid in the amino acid stretch encoded by SEQ ID No: 1 or SEQ ID No: 2 and/or (2) a substitution or a deletion in the position of Cys 31, Cys 32, or Cys 31 and Cys 32. In another embodiment, the present invention provides an expression system comprising a first DNA sequence coding on expression for a kosher animal casein kappa polypeptide comprising: (1) at least one altered amino acid in the amino acid stretch encoded by SEQ ID No: 1 or SEQ ID No: 2 and/or (2) a substitution or a deletion in the position of Cys 31, Cys 32, or Cys 31 and Cys 32. In another embodiment, the present invention provides an expression system comprising a first DNA sequence coding on expression for a kosher animal casein polypeptide comprising: (1) at least one altered amino acid in the amino acid stretch encoded by SEQ ID No: 1 or SEQ ID No: 2 and/or (2) a substitution or a deletion in the position of Cys 31, Cys 32, or Cys 31 and Cys 32 and (3) a second DNA sequence preceding the first sequence (5′ to the first DNA sequence). In another embodiment, the present invention provides that the second DNA sequence is operably linked in first DNA sequence. In another embodiment, the present invention provides that the second DNA sequence encodes a secretory signal polypeptide functional in mammalian cells. In another embodiment, the present invention provides that the expression system further comprises a promoter. In another embodiment, the present invention provides that the promoter is a milk protein gene promoter. In another embodiment, the present invention provides that the expression system is a replicable expression vector. In another embodiment, the present invention provides that the expression system comprises at least one intron. In another embodiment, the present invention provides that the intron is a kappa casein intron. In another embodiment, the present invention provides that the expression system comprising an altered kosher casein comprises a polypeptide comprising an amino acid sequence as set fourth in SEQ ID No: 4, 5, 7, 8, 10, 11, 13, or 14. In another embodiment, the present invention provides that the expression system comprising an altered kosher casein comprises a polypeptide comprising a substitution or a deletion in the position of Cys 31, Cys 32, or Cys 31 and Cys 32. In another embodiment, the present invention provides that the expression system comprising an altered kosher casein comprises a polypeptide comprising: (1) an amino acid sequence as set fourth in SEQ ID No: 4, 5, 7, 8, 10, 11, 13, or 14 and (2) a substitution or a deletion in the position of Cys 31, Cys 32, or Cys 31 and Cys 32.

In another embodiment, the present invention provides that the expression system is comprised within a cell. In another embodiment, the present invention provides that the expression system is comprised within a cell in a transgenic kosher mammal. In another embodiment, the present invention provides that the transgene encoding an altered kosher casein polypeptide, comprises a DNA sequence encoding at least one altered amino acid in the amino acid stretch encoded by SEQ ID No: 1 or SEQ ID No: 2. In another embodiment, the present invention provides that the transgene encoding an altered kosher casein polypeptide, comprises a DNA sequence encoding a substitution or a deletion in the position of Cys 31, Cys 32, or Cys 31 and Cys 32. In another embodiment, the present invention provides that the transgene encoding an altered kosher casein polypeptide, comprises a DNA sequence encoding: (1) at least one altered amino acid in the amino acid stretch encoded by SEQ ID No: 1 or SEQ ID No: 2 and (2) a substitution or a deletion in the position of Cys 31, Cys 32, or Cys 31 and Cys 32.

In another embodiment, the present invention provides that the transgene encoding an altered kosher casein polypeptide, comprises a DNA sequence encoding a kosher kappa casein deletion mutant. In another embodiment, the present invention provides that the transgene encoding an altered kosher casein polypeptide, comprises a DNA sequence encoding a kosher kappa casein wherein SEQ ID No: 1 or SEQ ID No: 2 is deleted and/or Cys 31, Cys 32, or Cys 31 and Cys 32 is substituted or a deleted from the DNA sequence encoding a kosher kappa casein. In another embodiment, the present invention provides that the transgene encodes the amino acid sequence as set fourth in SEQ ID No: 4, 5, 7, 8, 10, 11, 13, or 14. In another embodiment, the present invention provides that the transgene encodes an amino acid sequence of a kosher kappa casein comprising a substitution or a deletion in the position of Cys 31, Cys 32, or Cys 31 and Cys 32.

In another embodiment, the present invention provides that the expression system is an expression system comprising a 5′-flanking sequence from a milk protein gene of a mammal and a DNA sequence encoding a polypeptide having the amino acid sequence of a kosher kappa casein wherein (1) SEQ ID No: 1 or SEQ ID No: 2 is deleted and/or (2) Cys 31, Cys 32, or Cys 31 and Cys 32 is substituted or deleted from the DNA sequence. In another embodiment, the present invention provides that the flanking sequence is being capable of mediating expression of the polypeptide.

In another embodiment, the present invention provides that the expression system comprises at least one permissive RNA splice signal. In another embodiment, the present invention provides that the intron sequence or sequences is a mammal kappa casein intron. In another embodiment, the present invention provides that the intron sequence or sequences is a kosher mammal kappa casein intron. In another embodiment, the present invention provides that the intron sequence or sequences is a non-kosher mammal kappa casein intron.

In another embodiment, the present invention provides that the mammalian expression system according to the invention is one in which the DNA sequence is combined with regulatory element of a gene encoding a milk protein of a mammal so as to form a hybrid gene which is expressible in the mammary gland of an adult female of a non-human mammal harboring the hybrid gene so that the polypeptide encoded by the DNA sequence is produced when the hybrid gene is expressed. In another embodiment, the present invention provides that the mammalian expression system according to the invention is one in which the DNA sequence is combined with regulatory element of a gene encoding a milk protein of a mammal so as to form a hybrid gene which is expressible in the mammary gland of an adult female of a kosher mammal harboring the hybrid gene so that the polypeptide encoded by the DNA sequence is produced when the hybrid gene is expressed.

In another aspect, the present invention relates to a DNA sequence comprising an altered kosher kappa casein gene or an effective subsequence thereof containing elements capable of expressing a polypeptide having the activity of kappa casein or a digestive fragment thereof, or an analogue of said DNA sequence which: 1) hybridizes with the DNA sequence encoding the amino acid sequence as set fourth in SEQ ID NO: 4, 5, 7, 8, 10, 11, 13, or 14 under stringent hybridization conditions or 2) encodes a polypeptide, the amino acid sequence of which is at least 98% homologous with the amino acid sequence shown in 4, 5, 7, 8, 10, 11, 13, or 14.

In another aspect, the present invention relates to a DNA sequence comprising an altered kosher kappa casein gene or an effective subsequence thereof containing elements capable of expressing a polypeptide having the activity of kappa casein or a digestive fragment thereof, or an analogue of said DNA sequence which: 1) hybridizes with the DNA sequence encoding the amino acid sequence of a kosher kappa casein comprising a substitution or a deletion in the position of Cys 31, Cys 32, or Cys 31 and Cys 32 under stringent hybridization conditions or 2) encodes a polypeptide, wherein the amino acid sequence of which is at least 98% homologous with the amino acid of a kosher kappa casein comprising a substitution or a deletion in the position of Cys 31, Cys 32, or Cys 31 and Cys 32.

In another aspect, the present invention relates to a DNA sequence comprising an altered kosher kappa casein gene or an effective subsequence thereof containing elements capable of expressing a polypeptide having the activity of kappa casein or a digestive fragment thereof, or an analogue of said DNA sequence which: 1) hybridizes with the DNA sequence encoding the amino acid sequence as set fourth in SEQ ID NO: 4, 5, 7, 8, 10, 11, 13, or 14 wherein Cys 31, Cys 32, or Cys 31 and Cys 32 is deleted or substituted under stringent hybridization conditions or 2) encodes a polypeptide, wherein the amino acid sequence of which is at least 98% homologous with the amino acid sequence shown in 4, 5, 7, 8, 10, 11, 13, or 14 wherein Cys 31, Cys 32, or Cys 31 and Cys 32 is deleted or substituted.

In another aspect, the present invention relates, inter alia, to a DNA sequence encoding a polypeptide having the amino acid sequence as set fourth in SEQ ID No: 4, 5, 7, 8, 10, 11, 13, or 14 or the amino acid sequence of a kosher kappa casein wherein Cys 31, Cys 32, or Cys 31 and Cys 32 is deleted or substituted or the amino acid sequence as set fourth in SEQ ID No: 4, 5, 7, 8, 10, 11, 13, or 14 comprising a deletion or a substitution in Cys 31, Cys 32, or Cys 31 and Cys 32. In another aspect, the present invention relates, inter alia, to a DNA sequence which is an analogue or variant of the DNA sequences as describes hereinabove which has a biological activity of a kosher kappa casein. In one embodiment the DNA sequence comprises at least one intron sequence; in another embodiment the DNA sequence contains at least one permissive RNA splice signal.

In another aspect, the present invention provides additional regulatory or expression regulation sequences in addition to controlling transcription also contribute to RNA stability and processing, at least to the extent they are also transcribed.

Such expression regulation sequences are chosen to produce tissue-specific or cell type-specific expression of the recombinant DNA. Once a tissue or cell type is chosen for expression, 5′ and optional 3′ expression regulation sequences are chosen. Generally, such expression regulation sequences are derived from genes that are expressed primarily in the tissue or cell type chosen. Preferably, the genes from which these expression regulation sequences are obtained are expressed substantially only in the tissue or cell type chosen, although secondary expression in other tissue and/or cell types is acceptable if expression of the recombinant DNA in the transgene in such tissue or cell type is not detrimental to the transgenic animal. Particularly preferred expression regulation sequences are those endogenous to the species of animal to be manipulated. However, expression regulation sequences from other species such as those from human genes may also be used. In some instances, the expression regulation sequences and the structural DNA sequences (either genomic or cDNA) are from the same species, e.g. each from bovine species or from a human source. In such cases, the expression regulation sequence and the DNA sequence are homologous to each other. Alternatively, the expression regulation sequences and DNA sequences (either cDNA or genomic) are obtained from different species, e.g. an expression regulation sequence from bovine species and a DNA sequence from a human source. In such cases, the expression regulation and DNA sequence are heterologous to each other. The following defines expression regulation sequences from endogenous genes. Such definitions are also applicable to expression regulation sequences from non-endogenous, heterologous genes.

In another embodiment, the 5′ expression regulation sequence includes the transcribed portion of the endogenous gene upstream from the translation initiation sequence (the 5′ untranslated region or 5′ UTR) and those flanking sequences upstream therefrom which comprise a functional promoter. As used herein, a “functional promoter” includes those necessary untranscribed DNA sequences which direct the binding of RNA polymerase to the endogenous gene to promote transcription. Such sequences typically comprise a TATA sequence or box located generally about 25 to 30 nucleotides from the transcription initiation site. In another embodiment, the TATA box is also sometimes referred to as the proximal signal. In many instances, the promoter further comprises one or more distal signals located upstream from the proximal signal (TATA box) which are necessary to initiate transcription. In another embodiment, such promoter sequences are generally contained within the first 100 to 200 nucleotides located upstream from the transcription initiation site, but may extend up to 500 to 600 nucleotides or more from the transcription initiation site. In another embodiment, such sequences are either readily apparent to those skilled in the art or readily identifiable by standard methods. Such promoter sequences alone or in combination with the 5′ untranslated region are referred to herein as “proximal 5′ expression regulation sequences”.

In another embodiment, additional 5′flanking sequences (referred to herein as “distal 5′ expression regulation sequences”) are also included in the transgene. In another embodiment, such distal 5′ expression regulation sequences are believed to contain one or more enhancer and/or other sequences which facilitate expression of the endogenous gene and as a consequence facilitate the expression of the structural DNA sequence operably linked to the distal and proximal 5′ expression regulation sequences. In another embodiment, these 5′ expression regulation sequences regulate the spatial and temporal distribution of gene expression. In another embodiment, the amount of distal 5′ expression regulation sequences depends upon the endogenous gene from which the expression regulation sequences are derived. In another embodiment, such sequences comprise 5′flanking regions of approximately 1 kb, more preferably 16 kb and most preferably about 30 kb of 5′flanking sequence.

In another embodiment, the determination of the optimal amount of distal 5′ expression regulation sequences used from any particular endogenous gene is readily determined by varying the amount of distal 5′ expression regulation sequence to obtain maximal expression. In another embodiment, the distal 5′ expression regulation sequence will not be so large as to extend into an adjacent gene and will not include DNA sequences which adversely effect the level of transgene expression.

In another embodiment, a 3′ expression regulation sequences are included to supplement tissue or cell-type specific expression. In another embodiment, such 3′ expression regulation sequences include 3′ proximal and 3′ distal expression regulation sequences from an appropriate endogenous gene. In another embodiment, the 3′ proximal expression regulation sequences include transcribed but untranslated DNA positioned downstream from the translation stop signal in the recombinant DNA sequence (also referred to as the 3′ untranslated region or 3′ UTR). Such sequences generally terminate at a polyadenylation sequence (either from the endogenous gene or from other sources such as SV40) and sequences that may affect RNA stability. In another embodiment, 3′ UTR's comprise about 100 to 1000 nucleotides or more downstream from the translation stop signal in the gene from which the 3′ regulation sequence is derived. Distal 3′ expression regulation sequences include flanking DNA sequences downstream from the proximal 3′ expression regulation sequence. In another embodiment, some distal sequences are transcribed, but do not form part of the mRNA while other sequences in this 3′ distal expression regulation sequence are not transcribed at all. In another embodiment, distal 3′ expression regulation sequences comprise an enhancer and/or other sequences which enhance expression. In another embodiment, 3′ distal expression regulation sequences are necessary for efficient polyadenylation and contain transcription termination sequences. In another embodiment, such sequences comprise about 1-2 kb of 3′flanking sequence. In another embodiment, such sequences comprise about 3-8 kb of 3′flanking sequence. In another embodiment, such sequences comprise about 8-20 kb of 3′flanking sequence.

Although the use of both 5′ and 3′ expression regulation sequences are preferred, in some embodiments of the invention, endogenous 3′ regulation sequences are not used. In such cases, the 3′ proximal expression regulation sequences normally associated with the genomic DNA encoded by the recombinant DNA sequence are used to direct polyadenylation. In another embodiment, distal 3′ regulation sequences from the genomic DNA encoding the recombinant polypeptide are employed preferably in the same amounts as set forth for endogenous 3′ expression regulation sequences. In another embodiment, the recombinant polypeptide encoded by the transgene comprises either genomic DNA or a double stranded DNA derived from cDNA. As with the 5′ expression regulation sequences, the optimal amount of 3′ expression regulation sequence may be readily determined by varying the amount of 3′flanking sequence to obtain maximal expression of the recombinant polypeptide. In another embodiment, the distal 3′ regulation sequence, be it from an endogenous gene or a heterologous gene, will not extend into the adjacent gene from which it is derived and will exclude any sequences which adversely effect the level of transgene expression.

In addition to the 5′ and 3′ expression regulation sequences and the recombinant DNA (either genomic or derived from cDNA) the transgenes of the invention, in some embodiments, further comprise an intron sequence which interrupts the transcribed region of the transgene. In another embodiment, recombinant intervening sequences comprise a “hybrid intervening sequence”. In another embodiment, hybrid intervening sequences comprise a 5′ RNA splice signal and 3′ RNA splice signal from intervening sequences from heterologous or homologous sources. In another embodiment, such hybrid intervening sequences containing permissive RNA splice signals are used when the recombinant DNA corresponds to a cDNA sequence.

In another embodiment, the present invention comprises technology for cloning and manipulating DNA, the construction and microinjection of transgenes. In another embodiment, the transgenes of the invention, especially those having a length greater than about 50 kb, may be readily generated by introducing two or more overlapping fragments of the desired transgene into an embryonal target cell. In another embodiment, when so introduced, the overlapping fragments undergo homologous recombination which results in integration of the fully reconstituted transgene in the genome of the target cell.

In another embodiment, the present invention comprises homologous recombination in embryonic lung fibroblasts. In another embodiment, the present invention provides that embryonic lung fibroblasts are used as a source for nuclear transfer into enucleated oocyte. In another embodiment, the present invention provides that only nuclear DNA derived from the desired recombinant is utilized to create a transgenic animal (Brophy et. al. 2003).

In another embodiment, the present invention further provides manipulation of the glycosylation sites on the altered kosher casein. In another embodiment, these alteration are done by altering the genome of the host organism, for example a host cell or a transgenic animal, by introduction of recombinant genetic elements. In another embodiment, the present invention provides that these genetic elements can either encode additional or modified glycosyltransferases or other involved enzymes, and mediate their expression, or inhibit the function of endogenous glycosyltransferases or other involved enzymes.

The terms “sequence”, “subsequence”, “analogue” and “polypeptide” as used herein with respect to sequences, subsequences, analogues and polypeptides according to the invention should of course be understood as not comprising these phenomena in their natural environment, but rather, e.g., in isolated, purified, in vitro or recombinant form. When reference is made to a DNA sequence of the invention this should be understood to include “analogues”, “subsequences” and “modified sequences” as defined above Similarly, when reference is made to “a polypeptide of the invention” this should be understood to include any of the polypeptides defined in the following.

In the present context, the term “replicable” means that the vector is able to replicate in a given type of host cell into which it has been introduced Immediately upstream of the altered kosher kappa casein DNA sequence there may be provided a sequence coding for a signal polypeptide, the presence of which ensures secretion of the altered kosher kappa casein expressed by host cells harboring the vector. The signal sequence may be the one naturally associated with the kosher kappa casein DNA sequence or of another origin.

In another embodiment, the present invention further relates to a cell harboring a replicable expression vector. In another embodiment, this cell may be of any type of cell, i.e. a prokaryotic cell such as a bacterium, e.g. E. coli, a unicellular eukaryotic organism, a fungus or yeast, e.g. Saccharomyces cerevisiae or a cell derived from a multicellular organism, e.g. a mammal. In another embodiment, the mammalian cells are especially suitable for the purpose.

In another embodiment, the invention relates to a method of producing recombinant altered kosher kappa casein, in which a DNA sequence encoding altered kosher kappa casein is inserted in a vector which is able to replicate in a specific host cell, the resulting recombinant vector is introduced into a host cell which is grown in or on an appropriate culture medium under appropriate conditions for expression of altered kosher kappa casein and altered kosher kappa casein is recovered. In another embodiment, the invention provided that the medium used to grow the cells may be any conventional medium suitable for the purpose.

In another embodiment, altered kosher kappa casein is produced intracellularly by the recombinant host, that is, not secreted by the cell, it may be recovered by standard procedures comprising cell disrupture by mechanical means, e.g. sonication or homogenization, or by enzymatic or chemical means followed by purification.

In another embodiment, the DNA sequence encoding altered kosher kappa casein is preceded by a sequence coding for a signal polypeptide, the presence of which ensures secretion of altered kosher kappa casein from the cells so that at least a significant proportion of the altered kosher kappa casein expressed is secreted into the culture medium and recovered.

In another embodiment, the present invention relates to an altered kosher kappa casein (recombinant) polypeptide in which at least one amino acid residue has been modified by post-translational modification such as glycosylation.

In another embodiment, the present invention relates to a mammalian expression system comprising a DNA sequence encoding an altered kosher kappa casein inserted into a gene encoding a milk protein of a mammal so as to form a hybrid gene which is expressible in the mammary gland of an adult female of a kosher mammal harboring the hybrid gene.

In another embodiment, the present invention provides that the mammary gland as a tissue of expression and genes encoding milk proteins are generally considered to be particularly suitable for use in the production of heterologous proteins in transgenic kosher mammals as milk proteins are naturally produced at high expression levels in the mammary gland.

In another embodiment, the term “hybrid gene” denotes a DNA sequence comprising on the one hand a DNA sequence encoding altered kosher kappa casein as defined above and on the other hand a DNA sequence of the milk protein gene which is capable of mediating the expression of the hybrid gene product. The term “gene encoding a milk protein” or “milk protein gene” denotes an entire gene-altered kosher kappa casein, as well as an effective subsequence thereof capable of mediating and targeting the expression of the hybrid gene to the tissue of interest, i.e. the mammary gland.

In another embodiment, the hybrid gene is preferably formed by inserting in vitro the DNA sequence encoding altered kosher kappa casein into the milk protein gene by use of techniques known in the art. Alternatively, the DNA sequence encoding altered kosher kappa casein can be inserted in vivo by homologous recombination.

In another embodiment, the DNA sequence encoding altered kosher kappa casein will be inserted in one of the first exons of the milk protein gene of choice or an effective subsequence thereof comprising the first exons and preferably a substantial part of the 5′ flanking sequence which is believed to be of regulatory importance.

In another embodiment, the hybrid gene preferably comprises a sequence encoding a signal polypeptide so as to enable the hybrid gene product comprising an altered kosher kappa casein to be secreted correctly into the mammary gland. In another embodiment, the signal polypeptide will typically be the one normally found in the milk protein gene in question or one associated with the DNA sequence encoding altered kosher kappa casein.

In another embodiment, other signal sequences capable of mediating the secretion of the hybrid gene comprising an altered kosher kappa casein product to the mammary gland are relevant. In another embodiment, various elements of the hybrid gene should be fused in such a manner as to allow for correct expression and processing of the gene product. In another embodiment, the DNA sequence encoding the signal polypeptide of choice should be precisely fused to the N-terminal part of the DNA sequence encoding altered kosher kappa casein. In another embodiment, in the hybrid gene comprising an altered kosher kappa casein, the DNA sequence encoding altered kosher kappa casein will normally comprise its stop codon, but not its own message cleavance and polyadenylation site. Downstream of the DNA sequence encoding altered kosher kappa casein, the mRNA processing sequences of the milk protein gene will normally be retained.

In another embodiment, the DNA sequence encoding altered kosher kappa casein to be inserted in the expression system of the invention may be of cDNA, genomic or synthetic origin or any combination thereof. In another embodiment, cDNA molecular encoding altered kosher kappa casein of the invention is obtained by performing RT-PCR with specific kappa casein primers on mRNA. In another embodiment, cDNA molecular encoding altered kosher kappa casein of the invention is obtained by performing RT-PCR with specific kappa casein primers on isolated mRNA by methods known to one of skill in the art. While some expression systems have been found to function best when cDNA encoding a desirable protein is used, others have been found to require the presence of introns and other regulatory regions in order to obtain a satisfactory expression (Hennighausen et al. 1990). In some cases it may be advantageous to introduce genomic structures in vector constructs compared to cDNA elements (Brinster et al. 1988). The intron and exon structure may result in higher steady state mRNA levels than obtained when cDNA based vectors are used.

In the specification, the term “intron” includes the whole of any natural intron or part thereof.

In another embodiment, the present invention relates to a method of producing a transgenic kosher mammal capable of expressing an altered kosher kappa casein, comprising injecting an expression system of the invention as defined above into a fertilized egg or a cell of an embryo of a mammal so as to incorporate the expression system into the germline of the mammal and developing the resulting injected fertilized egg or embryo into an adult female mammal.

In another embodiment, the present invention relates to a method of producing a transgenic kosher mammal capable of expressing a polypeptide having the amino acid sequence SEQ ID NO: 4, 5, 7, 8, 10, 11, 13, 14 or an analogue or variant thereof which has a biological activity of an altered kosher kappa casein, wherein the method comprises chromosomally incorporating a DNA sequence encoding the polypeptide into the genome of a kosher mammal.

In another embodiment, the present invention relates to a method comprising injecting an expression system encoding an altered kosher kappa casein or an analogue, variant or subsequence thereof into a fertilized egg or a cell of an embryo of a mammal so as to incorporate the expression system into the germline of the mammal and developing the resulting injected fertilized egg or embryo into an adult kosher female mammal.

In another embodiment, the present invention relates to a method comprising 1) destroying the endogenous kappa casein polypeptide expressing capability of the mammal so that substantially no endogenous polypeptide is expressed and inserting an expression system of the invention into the germline of the mammal in such a manner that the polypeptide comprising an altered kosher kappa casein amino acid sequence or an analogue or variant thereof which has a biological activity of an altered kosher kappa casein, is expressed in the mammal and/or 2) replacing the gene encoding the endogenous polypeptide or part thereof with an expression system of the invention thereby making the kosher mammal substantially incapable of expressing the corresponding endogenous polypeptide.

In another embodiment, the present invention provides that desirable phenotypes for transgenic kosher mammals include, but are not limited to, the production of recombinant altered kosher kappa casein polypeptides of the invention in the milk of female transgenic kosher mammal.

In another embodiment, the present invention provides that the transgenic kosher mammal of the invention is produced by introducing a “transgene” into an embryonal target cell of the animal of choice. In one aspect of the invention, a transgene is a DNA sequence which is capable of producing a desirable phenotype when contained in the genome of cells of a transgenic kosher mammal. In another embodiment, the transgene comprises a “recombinant DNA sequence” encoding a “recombinant altered kosher kappa casein polypeptide”. In another embodiment, the transgene is capable of being expressed to produce the recombinant polypeptide.

In another embodiment, the incorporation of the expression system into the germline of the mammal may be performed using any suitable technique, e.g. as described in Hogan B., Constantini, F. and Lacy, E. Manipulating the Mouse Embryo. A Laboratory Manual. Cold Spring Harbor Laboratory Press, 1986 or in WO91/08216.

In another embodiment, methods of introducing transgenes or overlapping transgene fragments into embryonal target cells include microinjection of the transgene into the pronuclei of fertilized oocytes or nuclei of ES cells of the non-human animal. Such methods for murine species are well known to those skilled in the art.

In another embodiment, the transgene is introduced into an animal by infection of zygotes with a retrovirus containing the transgene (Jaenisch, R. (1976), Proc. Natl. Acad. Sci. USA, 73, 1260-1264). In another embodiment, the method is microinjection of the fertilized oocyte. In another embodiment, the fertilized oocytes are first microinjected by standard techniques. In another embodiment, they are thereafter cultured in vitro until a “pre-implantation embryo” is obtained. Such pre-implantation embryos preferably contain approximately 16 to 150 cells. In another embodiment, the 16 to 32 cell stage of an embryo is commonly referred to as a morula. In another embodiment, those pre-implantation embryos containing more than 32 cells are commonly referred to as blastocysts. In another embodiment, they are generally characterized as demonstrating the development of a blastocoel cavity typically at the 64 cell stage.

In another embodiment, methods for culturing fertilized oocytes to the pre-implantation stage include those described by Gordon et al. (1984), Methods in Enzymology, 101, 414; Hogan et al. (1986) in Manipulating the Mouse Embryo, Cold Spring Harbor Laboratory Press, Cold Spring Harbor, N.Y. (for the mouse embryo); and Hammer et al. (1985), Nature, 315, 680 (for rabbit and porcine embryos); Gandolfi et al. (1987) J. Reprod. Fert. 81, 23-28; Rexroad et al. (1988) J. Anim. Sci. 66, 947-953 (for ovine embryos); and Eyestone, W. H. et al. (1989) J. Reprod. Fert. 85, 715-720; Camous et al. (1984) J. Reprod. Fert. 72, 779-785; and Heyman, Y. et al. (1987) Theriogenology 27, 5968 (for bovine embryos). In another embodiment, pre-implantation embryos are thereafter transferred to an appropriate female by standard methods to permit the birth of a transgenic or chimeric animal depending upon the stage of development when the transgene is introduced. In another embodiment, mosaic animals can be bred to form true germline transgenic animals.

In another embodiment, the detection of transgene integration in the pre-implantation embryo is highly desirable. In another embodiment, methods are provided for identifying embryos wherein transgenesis has occurred and which permit implantation of transgenic embryos to form transgenic animals. In another embodiment, one or more cells are removed from the pre-implantation embryo. In another embodiment, when equal division is used, the embryo is preferably not cultivated past the morula stage (32 cells). Division of the pre-implantation embryo (reviewed by Williams et al. (1984) Theriogenology 22, 521-531) results in two “hemi-embryos” (hemi-morula or hemi-blastocyst) one of which is capable of subsequent development after implantation into the appropriate female to develop in utero to term. Although equal division of the pre-implantation embryo is preferred, it is to be understood that such an embryo may be unequally divided either intentionally or unintentionally into two hemi-embryos which are not necessarily of equal cell number. Essentially, all that is required is that one of the embryos which is not analyzed as hereinafter described be of sufficient cell number to develop to full term in utero. In a specific embodiment, the hemi-embryo which is not analyzed as described herein, if shown to be transgenic, is used to generate a clonal population of transgenic kosher animals.

In another embodiment, one of each of the hemi-embryos formed by division of pre-implantation embryos is analyzed to determine if the transgene has been integrated into the genome of the organism. In another embodiment, each of the other hemi-embryos is maintained for subsequent implantation into a recipient female of the species.

In another embodiment, the identification of the pre-implantation embryos containing the integrated transgene is achieved by analyzing the DNA from one of each of the hemi-embryos. In another embodiment, DNA is typically obtained by lysing the hemi-embryo and analyzing the thus released DNA. In another embodiment, a polymerase chain reaction is performed to amplify all or part of the transgene. In another embodiment, when the entire transgene is amplified, two extension primers each complementary to opposite strands at opposing ends of the transgene are used for amplification. In another embodiment, the amplified DNA from the hemi-embryo is subjected to electrophoresis followed by hybridization with labeled probe complementary to the region of the transgene between the two extension primers. In another embodiment, this facilitates the determination of the size of the amplified DNA sequences, if any, and provides an indication of whether the transgene has been integrated into the pre-implantation embryo from which the hemi-embryo is obtained (now called a “transgenic hemi-embryo”). In another embodiment, if it has, the remaining untreated transgenic hemi-embryo is transplanted into a recipient parent. In another embodiment, after in utero development, the transgenic non-human animal having the desired phenotype conferred by the integrated transgene is identified by an appropriate method in utero or after birth.

In another embodiment, the above described method for detecting transgenesis in pre-implantation embryos is combined with embryonic cloning steps to generate a clonal population of transgenic embryos which may thereafter be implanted into recipient females to produce a clonal population of transgenic kosher animals also having the same genotype. In another embodiment, it is to be understood that transgenic embryos and/or kosher transgenic animals having the same “genotype” means that the genomic DNA is substantially identical between the individuals of the embryo and/or transgenic animal population. In another embodiment, it is to be understood, however, that during mitosis various somatic mutations may occur which may produce variations in the genotype of one or more cells and/or animals. In another embodiment, a population having the same genotype may demonstrate individual or subpopulation variations.

In another embodiment, after a hemi-embryo is identified as a transgenic hemi-embryo, it is cloned. Such embryo cloning may be performed by several different approaches. In another embodiment, the transgenic hemi-embryo is cultured in the same or in a similar medium as used to culture individual oocytes to the pre-implantation stage. In another embodiment, the “transgenic embryo” so formed (preferably a transgenic morula) is then divided into “transgenic hemi-embryos” which can then be implanted into a recipient female to form a clonal population of two transgenic non-human animals In another embodiment, the two transgenic hemi-embryos obtained may be again cultivated to the pre-implantation stage, divided, and recultivated to the transgenic embryo stage. In another embodiment, this procedure is repeated until the desired number of clonal transgenic embryos having the same genotype is obtained. In another embodiment, transgenic embryos may then be implanted into recipient females to produce a clonal population of transgenic non-human animals.

In another embodiment, the transgenic embryo is cloned by nuclear transfer according to the techniques of Prather et al. (1987) Biol. Reprod. 37, 859-866; Roble et al. (1987) J. Anim Sci. 64, 642-664. In another embodiment, nuclei of the transgenic embryo are transplanted into enucleated oocytes, each of which is thereafter cultured to the blastocyst stage. In another embodiment, the transgenic embryos are resubjected to another round of cloning by nuclear transplantation or may be transferred to a recipient parent for production of transgenic offspring having the same genotype.

In another embodiment, other methods may be used to detect transgenesis. In another embodiment, In utero analysis is performed by several techniques. In another embodiment, transvaginal puncture of the amniotic cavity is performed under echoscopic guidance (Bongso et al. (1975) Vet. Res. 96, 124-126; Rumsey et al. (1974) J. Anim Sci. 39, 386-391). In another embodiment, this involves recovering about 15 to 20 milliliters of amniotic fluid between about day 35 and day 100 of gestation.

In another embodiment, fetal cells are recovered by chorion puncture. In another embodiment, this method is performed transvaginally and under echoscopic guidance.

In another embodiment, transgenesis is detected after birth. In another embodiment, a transgene integration is detected by taking an appropriate tissue biopsy such as from the ear or tail of the putative kosher transgenic animal.

In another embodiment, the integrated in the germ line, the DNA sequence encoding an altered kosher kappa casein is expressed at high levels to produce a correctly processed and functional altered kosher kappa casein. In another embodiment, transgenic females from which recombinant polypeptide can be harvested can thus be bred in the following generations.

In another embodiment, the present invention provides a transgenic kosher mammal, comprising a transgene encoding a non-kosher mammal casein. In another embodiment, the present invention provides a transgenic kosher mammal, comprising a transgene encoding a camel casein. In another embodiment, the present invention provides a transgenic kosher mammal, comprising a transgene encoding a horse casein. In another embodiment, the present invention provides a transgenic kosher mammal, comprising a transgene encoding a pig casein. In another embodiment, the present invention provides a transgenic kosher mammal, comprising a transgene encoding a human casein.

In another embodiment, the present invention provides a transgenic kosher mammal, comprising a transgene encoding a modified kosher casein polypeptide of the invention. In another embodiment, the invention provides a composition comprising casein derived from a transgenic kosher mammal which comprises a transgene encoding a non-kosher mammal casein.

In another embodiment, the invention provides a composition comprising an altered kosher animal casein polypeptide, comprising: (a) at least one altered amino acid in a defined amino acid stretch encoded by SEQ ID No: 1 or SEQ ID No: 2, or (b) a substitution or a deletion in the position of Cys 31, Cys 32, or both Cys 31 and Cys 32; or (c) at least one altered amino acid in a defined amino acid stretch encoded by SEQ ID No: 1 or SEQ ID No: 2 and a substitution or a deletion in the position of Cys 31, Cys 32, or both Cys 31 and Cys 32. In another embodiment, the invention provides a composition comprising an altered kosher animal casein of the invention. In another embodiment, the invention provides that the composition comprising an altered kosher animal casein of the invention is produced in an animal. In another embodiment, the invention provides that the composition comprising an altered kosher animal casein of the invention is produced in a transgenic animal. In another embodiment, the invention provides that the composition comprising an altered kosher animal casein of the invention is produced in a kosher transgenic animal. In another embodiment, the invention provides that milk comprising an altered kosher animal casein of the invention is produced in a transgenic animal. In another embodiment, the invention provides that milk comprising an altered kosher animal casein of the invention is produced in a transgenic kosher animal.

In another embodiment, a composition comprising an altered kosher animal casein of the invention is characterized by reduced allergenicity to a kosher casein when consumed by a human subject allergic to wild-type kosher casein. In another embodiment, a composition comprising an altered kosher animal casein of the invention is characterized by reduced allergenicity to a kosher casein when in contact with skin of a human subject allergic to wild-type kosher casein.

In another embodiment, a mutated kappa-casein or altered kappa casein as described herein is used for the induction of desensitization and/or oral tolerance. In another embodiment, blocking IgG antibodies and/or Treg cells against a kappa-casein are produced without the risk of an allergic reaction.

In another embodiment, a mutated kappa-casein or altered kappa casein as described herein is used as a marker on Skin Prick Test for those who would remain allergic and may not become tolerant as they grow older.

In another embodiment, milk of the present invention is a food product for humans of all ages derived from other animals. In another embodiment, milk of the present invention is a food product for human infants. In another embodiment, milk of the present invention is mammalian milk. In another embodiment, milk of the present invention is produced on an industrial scale. In another embodiment, milk of the present invention is produced in cattle such as but not limited to the Holstein, Ayrshire, Brown Swiss, Guernsey, Jersey, and Milking Shorthorn.

In another embodiment, milk of the present invention is an emulsion of butterfat globules within a water-based fluid. In another embodiment, milk of the present invention is unhomogenized. In another embodiment, milk of the present invention is homogenized. In another embodiment, milk of the present invention comprises vitamins A, D, E, K, or a combination thereof. In another embodiment, the composition of the present invention is cream. In another embodiment, cream is derived from milk.

In another embodiment, milk of the present invention comprises calcium. In another embodiment, milk of the present invention comprises iodine. In another embodiment, milk of the present invention comprises Vitamin B12. In another embodiment, milk of the present invention comprises riboflavin. In another embodiment, milk of the present invention comprises Biotin and pantothenic acid. In another embodiment, milk of the present invention comprises potassium and magnesium. In another embodiment, milk of the present invention comprises Selenium. In another embodiment, milk of the present invention comprises Thiamine. In another embodiment, milk of the present invention comprises conjugated linoleic acid.

In another embodiment, the altered kosher casein of the invention is used in the manufacture of adhesives. In another embodiment, the altered kosher casein of the invention is used in the manufacture of binders. In another embodiment, the altered kosher casein of the invention is used in the manufacture of protective coatings. In another embodiment, the altered kosher casein of the invention is used in the manufacture of plastics (such as for knife handles and knitting needles). In another embodiment, the altered kosher casein of the invention is used in the manufacture of fabrics. In another embodiment, the altered kosher casein of the invention is used by bodybuilders as a slow-digesting source of amino acids as opposed to the fast-digesting whey protein, and also as an extremely high source of glutamine (post-workout).

In another embodiment, the present invention further provides an infant formula comprising an altered kosher kappa casein, in particular an altered kosher kappa casein polypeptide of the invention as defined above.

In another embodiment, modified casein of the present invention is used in pharmaceutical compositions. In another embodiment, modified casein of the present invention is used in pharmaceutical compositions as a binder. In another embodiment, pharmaceutical compositions comprising casein of the invention are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Topical formulations include, in another embodiment, gels, ointments, creams, lotions, drops and the like.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active agent over a period of time.

In another embodiment, pharmaceutical compositions comprising casein of the invention further comprise parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e. compositions in which the active compound is released over a period of time after administration. Controlled- or sustained-release compositions include formulation in lipophilic depots (e.g. fatty acids, waxes, oils). In another embodiment, the composition is an immediate-release composition, i.e. a composition in which of the active compound is released immediately after administration.

In another embodiment, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the target cell, thus requiring only a fraction of the systemic dose (see, e.g., Goodson, in Medical Applications of Controlled Release, supra, vol. 2, pp. 115-138 (1984); and Langer R, Science 249: 1527-1533 (1990).

In another embodiment, the present invention provides a kit comprising a reagent utilized in performing a method of the present invention. In another embodiment, the present invention provides a kit comprising a composition, tool, or instrument of the present invention.

### EXPERIMENTAL DETAILS SECTION MATERIALS AND EXPERIMENTAL METHODS

### Patients

The study protocol was approved by the Institutional Review Board at the Medical Center of Assaf Harofeh Hospital according to the Helsinki Declaration. Evaluations were performed in a referral center for allergic disorders at the investigator's institution.

### Milk

Fresh milk from lactating pig was obtained from Kibbutz Lehavim (Israel). Milk from deer and ibex was obtained from lactating animals at Odem Farms (Israel). These animals were milked specifically for the study. Milk from camel was obtained from R. Yagil Ph.D. (Beersheva) and milk from buffalo from Moshav Bitzaron (Israel). Fresh milk of the studied species was placed at 4 oC and used within five days.

### Skin Prick Test (SPT)

SPTs were performed through a standard technique with a Wyeth needle (Heinz Herenz, Hamburg, Germany). Allergy extracts to cow milk protein and soy were purchased from ALK-Abelló, (Port Washington, N.Y.). Milk from different species, commercial allergy extracts, histamine at 1 mg/ml (Center Laboratories, Port Washington, N.Y.) and negative controls (saline) were applied. The mean diameters of the wheal and erythema (flare) were measured after 15 minutes (a planimeter was used to measure the area of the papule). If the wheal or flare diameter exceeded that of the histamine positive control the test was regarded as positive.

In total, thirty-two patients were evaluated by skin prick testing to the milks derived from the various species listed.

### Statistical Analysis

The Fisher exact test was used to determine whether pig's milk allergy was dependent on allergy to cow's milk.

### Example 1

### Reactivity to a “Kosher” Epitope

A total of 24 patients with known cow's milk allergy and a positive SPT to cow's milk protein were tested for cross-sensitization to milk proteins derived from deer, ibex, buffalo, pig and camel. The clinical characteristics of the twenty four subjects are listed in Table 1. Thirteen of the patients studied were male and eleven were female. Nineteen out of twenty four of the patients (79%) developed a rash after the ingestion of cow's milk, either as the sole symptom (n=2) or as part of a constellation of symptoms as listed in Table 1. With the exception of one patient (p#6), the time from ingestion to the onset of their clinical manifestations was less than twenty minutes. For patient #6, 120 minutes elapsed until clinical symptoms appeared. Eight out of 24 patients (33%) developed anaphylaxis. Six developed generalized edema and in one patient the edema was restricted to the ear. Sixteen out of 24 patients (67%) vomited as part of their clinical constellation of presenting symptoms. Only 5/24 (22%) of patients developed shortness of breath as part of their presenting symptoms. The age of onset was within the first nine months of life for all patients except for one (patient #11) who developed the allergy at an age of four years. All of these patients were still allergic to cow's milk as judged by incidental exposure.

TABLE 1
Clinical characteristics and symptoms associated
with cow's milk protein allergy

| | | Age | Age | Onset of | |
|---|---|---|---|---|---|
| Patient | | at onset | at exam | Reaction | Clinical |
| no. | Gender | (months) | (months) | (minutes) | Manifestations |
| 1 | M | 1 | 30 | 15 | R, V, |
| 2 | F | <1 | 13 | <5 | R, A, V |
| 3 | M | 5 | 180 | <5 | R, A, |
| 4 | M | 1.75 | 22 | <5 | R, V, SB |
| 5 | M | 6 | 31 | 10 | R, V |
| 6 | F | 4 | 26 | 120 | V |
| 7 | F | 6 | 22 | 10 | R, V |
| 8 | F | 1 | 2.5 | 20 | R, E |
| 9 | M | 9 | 20 | 5 | A, V, SB, E |
| 10 | M | 4 | 192 | 5 | R, E |
| 11 | M | 48 | 96 | 20 | R, |
| 12 | F | 2 | 174 | 5 | R, A |
| 13 | F | 6 | 48 | 5 | R, V |
| 14 | M | 2 | 47 | 20 | R, V, E |
| 15 | F | 3 | 96 | 5 | R, A, V, SB, E |
| 16 | M | 0.1 | 54 | 20 | R, V |
| 17 | M | 1 | 31 | 5 | R |
| 18 | F | 1 | 31 | 20 | R, V, A, SB |
| 19 | M | 6 | 108 | 5 | R, V, A, E |
| 20 | M | 6 | 20 | 20 | R, E¹ |
| 21 | M | 1.5 | 9 | 5 | R, A, * |
| 22 | F | 1.5 | 36 | 5 | V, SB |
| 23 | F | 6 | 39 | 5 | V |
| 24 | F | 2 | 42 | 5 | V |

R = rash;
V = Vomiting;
A = Anaphylaxis;
SB = Shortness of breath
E = Edema,
E¹ = edema localized to the ear,
* = Diarrhea

The results of the SPT are listed in Table 2, Table 3 lists the results of the control group. Reactivity to milk derived from the deer, ibex, buffalo, pig, and camel were tested. In addition, the response to commercial allergy extracts to cows milk and soy was evaluated. The response to histamine served as an internal positive control. All twenty-four patients who were positive to CMP were likewise positive to buffalo, deer and Ibex (p=0). Only five out of 24 (20.8%) of these patients, on the other hand, were positive to pig milk. One of the eight control patients had a positive SPT to pig milk but all of the control groups were negative to cow, ibex and deer milk (Table 3). Thus, there was no connection between skin prick positive to pig's milk and an allergy to CMP, using the Fisher exact test (p=0.52). Four out of twenty-three (17.4%) cross-sensitized to soy, three of whom were also positive to pig-derived proteins (patients #12, 16, 17). It is interesting to note, however, that all these patients with positive SPT to soy, by history tolerated oral soy without any clinical reaction. In general, the size of the wheal and flare to deer and ibex milk were large compared to the reaction to cow's milk extract (Table 2). Upon SPT examination of five patients to fresh cow milk, however, the size of the reaction was similar to that of the commercial extract (data not shown).

TABLE 2
SPT results in cow's milk protein allergic patients

| Patient | | | | | | | | |
|---|---|---|---|---|---|---|---|---|
| no. | Histamine | CM | Deer | Ibex | Buff | Pig | Camel | Soy |
| 1 | 2/3 | 6/12 | 7/12 | 3/5 | 8/12 | 0 | Nd | nd |
| 2 | 6/10 | 7/30 | 17/30 | 13/30 | 16/40 | 3/3 | Nd | 4/8 |
| 3 | 4/4 | 12/22 | 13/27 | 12/35 | 12/35 | 2/2 | Nd | 4/4 |
| 4 | 6/8 | 13/19 | 14/21 | 16/30 | 12/20 | 2/2 | Nd | 2/2 |
| 5 | 2/6 | 2/8 | 4/10 | 4/10 | 2/8 | 2/3 | Nd | 1/5 |
| 6 | 5/7 | 10/20 | 15/37 | 15/30 | 15/30 | 0 | Nd | 0 |
| 7 | 4/12 | 6/15 | 8/20 | 10/22 | 12/25 | 10/25 | Nd | 0 |
| 8 | 7/12 | 8/16 | 11/19 | 9/22 | 11/28 | 0 | Nd | 0 |
| 9 | 6/8 | 12/19 | 16/30 | 18/28 | 18/26 | 4/4 | Nd | 6/8 |
| 10 | 5/7 | 9/19 | 16/22 | 15/25 | 13/25 | 0 | Nd | 0 |
| 11 | 3/14 | 4/15 | 7/19 | 9/20 | 14/26 | 0/4 | Nd | 0/4 |
| 12 | 10/12 | 12/22 | 25/40 | 24/40 | 22/32 | 12/12 | Nd | 12/20 |
| 13 | 3/7 | 12/18 | 15/25 | 13/20 | 14/21 | 2/4 | Nd | 0 |
| 14 | 6/9 | 9/16 | 21/33 | 20/36 | 19/27 | 6/9 | Nd | 5/6 |
| 15 | 6/8 | 11/20 | 10/20 | 9/20 | 11/24 | 4/4 | Nd | 4/6 |
| 16 | 2/4 | 12/13 | 18/27 | 15/20 | 14/26 | 13/18 | Nd | 4/6 |
| 17 | 8/15 | 21/30 | 16/30 | 26/40 | 20/30 | 11/15 | 0 | 18/25 |
| 18 | 5/7 | 14/24 | 20/30 | 17/30 | 16/22 | 5/5 | 8/14 | 5/7 |
| 19 | 6/8 | 14/18 | 12/20 | 14/25 | 16/26 | 0 | 0 | 4/5 |
| 20 | 4/6 | 8/13 | 13/20 | 12/17 | 11/18 | 0 | 2/2 | 6/6 |
| 21 | 10/12 | 10/16 | 12/18 | 20/24 | 12/20 | 0 | 0 | 0 |
| 22 | 7/9 | 11/23 | 23/30 | 17/20 | 17/27 | 2/2 | 4/4 | 4/4 |
| 23 | 8/10 | 10/14 | 16/30 | 12/22 | 22/26 | 21/25 | 14/20 | 6/6 |
| 24 | 6/8 | 12/18 | 18/22 | 18/30 | 16/20 | 3/3 | 4/4 | 4/4 |
| Total | | 24/24 | 24/24 | 24/24 | 24/24 | 5/24 | 2/8 | 4/23 |

TABLE 3
SPT results in Control Group

| | | Age at | | | | | | | | |
|---|---|---|---|---|---|---|---|---|---|---|
| | | examination | | | | | | | | |
| | | (months) | Histamine | CM | Deer | Ibex | Buff | Pig | Camel | Soy |
| 1 | M | 17 | 10/20 | 0 | 0 | 0 | nd | 4/4 | nd | nd |
| 2 | F | 4 | 7/10 | 0 | 0 | 0 | nd | 0 | nd | nd |
| 3 | M | 30 | 4/6 | 0 | 0 | | nd | 0 | nd | nd |
| 4 | F | 192 | 3/5 | 0 | 3/3 | | nd | 4/12 | nd | nd |
| 5 | M | 36 | 3/4 | 0 | 3/3 | 0 | nd | 0 | nd | nd |
| 6 | F | 5 | 5/8 | 0 | 2/3 | 0 | nd | 0 | nd | nd |
| 7 | F | 30 | 4/4 | 0 | 0 | 0 | nd | 0 | nd | nd |
| 8 | F | 360 | 3/3 | 2/2 | 2/2 | 0 | nd | 2/1 | nd | nd |
| Total | | | | 0 | 0 | 0 | nd | 1/8 | | |

nd = no data

This is the first study that investigates the cross-sensitization of milk protein from deer, Ibex and pig in patients with CMP allergy. The study population consisted of patients with a convincing history of clinical milk allergy based on a positive SPT and a significant clinical reaction. In the majority of cases, the clinical reaction to the ingestion of milk included a rash appearing within 20 minutes from time of exposure, while in 3/24 (12.5%) patients, gastrointestinal symptoms predominated. A positive SPT in all our patients, however, ascertains that our patients have true IgE-mediated cow milk protein allergy, and not cow's milk intolerance (CMI). The latter, which can be classified into the group of food protein-induced enterocolitis syndrome (FPIES), is a clinical reaction that takes place 2-48 hours following the exposure and is confined to the gastrointestinal system with severe vomiting and/or diarrhea. This reaction is thought to be immunologically mediated but not via IgE, and the SPT is negative.

The significant clinical history to cow's milk along with the positive SPT and the size of the SPT ascertain clinical allergy to CMP. Similarly, the size of the SPT to deer and ibex in most of the patients suggests a likely clinical reaction to milk derived from these animals. The fact that all four patients that tested positively to soy extract tolerated oral soy, is consistent with other reports in the literature.

The immunogenic epitopes in milk causing IGE-mediated CMP allergy may be diverse, although as reported by Docena et al., anti-casein-specific IgE antibodies were present in all (80/80) sera examined from patients with a compatible history for CMP allergy and positive IgE against cow's milk protein (Docena, 1996). In contrast, only 10/80 showed reactivity to beta lactoglobulin. The presence of IgE antibodies against at least 1 of 3 epitopes present on either alpha(s1)-casein, alpha(s2)-casein, or kappa-casein identified all patients with persistent CMA. Furthermore, lymphocyte proliferative responses to the caseins were only observed in clinically reactive IgE-mediated CMP allergy, but not in non-IgE mediated cow's milk allergy. Additional evidence implicating the caseins in CMP allergy, is that a decreased proliferative response to kappa-casein noted in tolerized IgE-mediated CMP patients was abrogated by Treg removal. It was of interest, therefore, when we noted the reported phylogenetic relationship of beta-casein (exon 7) and kappa-casein (exon 4) among various mammals. The goat, sheep, cow, deer, giraffe and pronghorn are most closely related versus for example the camel, pig, tapir and zebra.

The casein genes from non-kosher animals are more closely related to the human derived casein sequence and in general, CMP allergic patients tolerate breast milk. Patients allergic to CMP are highly crossreactive to milk from sheep and goats but not to ass, mare or camel. The present example shows that patients with CMP allergy are also shown to be crossreactive to buffalo, deer (cervus aloposs) and Ibex milk but in the majority of cases not to pig or camel.

In summary, patient's with CMP allergy are cross-sensitized to milk derived from other kosher animal species but less so to the non-kosher animals tested. Thus, in addition to their external physical characteristics described in the Old Testament, kosher animals also share internal structural similarities in their milk components. These structural similarities have wide-ranging clinical implications regarding the type of milk that may be tolerated.

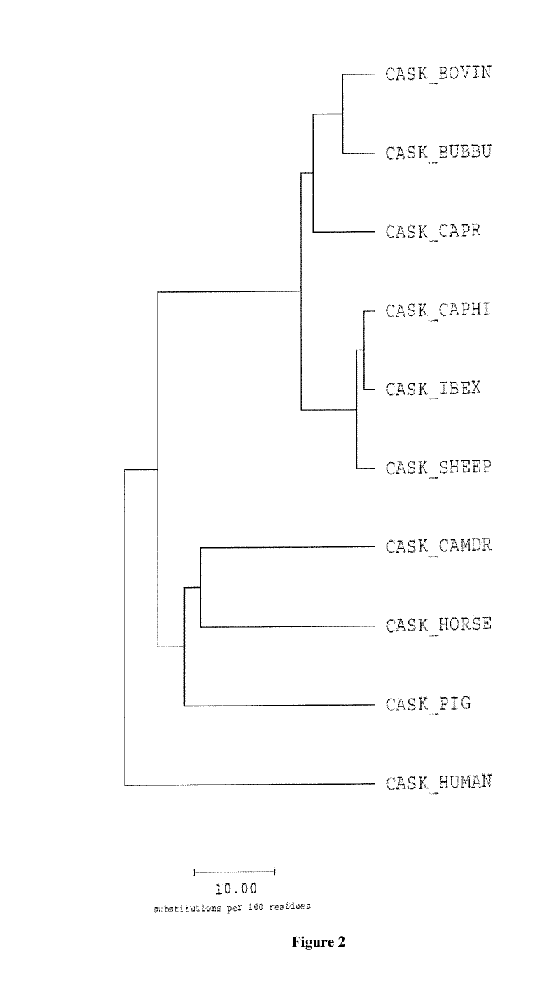

The phylogenetic tree showing the genetic distances in kappa casein among the different mammal species discussed hereinabove are presented in FIG. 2.

Analysis of the sequences presented in FIG. 1 revealed that kosher animal comprise either the AA sequence VPAKSCQD (SEQ ID No: 1) or VPAKSCQA (SEQ ID No: 2). None of these sequences is present in the AA sequence encoding kappa casein in non-kosher animals. The kappa casein in non-kosher animals was found to be less allergenic or non-allergenic. Thus, it is most likely that SEQ ID No: 1 or SEQ ID No: 2 are responsible to some extent for CMP and other related allergies.

### Example 2

### Cloning and Sequencing of CDNA Encoding Altered Kosher Kappa Casein

Construction of the expression systems of the invention, and the molecular biological characterization of it, employs standard methods generally known in the art of recombinant DNA.

cDNA of Kosher Kappa casein is prepared by methods known to one of skill in the art. Various isoforms of altered cDNA sequences of Kosher Kappa casein such as but not limited to the isoforms encoded by SEQ ID No: 4, 5, 7, 8, 10, 11, 13, and 14 are prepared by methods known to one of skill in the art.

The library of the isoforms of altered cDNA sequences of Kosher Kappa casein is screened by immunological methods using the kappa casein polyclonal antibodies.

The procedure used is as follows: E. coli Y1090 bacteria are grown on LA plates containing 50 μg/ml of carbenicillin. A single colony is isolated and grown over night in a LB containing 0.2% maltose and 10 mM MgSO₄. 0.4 ml of the culture is then mixed with diluted library phages and adsorption is allowed for 15 min at 37° C. The infected culture is mixed with 7 ml soft agarose (0.75% agarose in LB and 10 mM MgSO.sub.4). The soft agarose mixture is poured on 150 mm LA plates. The plates are incubated at 42° C. C. for about 3.5 h, until plaques are visible. Thereafter, each plate is overlayed with a membrane (DuPont NEN, Colony Plaque Screen) previously saturated in 10 mM IPTG (Isopropyl-.beta.-D-thiogalactoside), and incubated over night at 37° C. The positions of the membranes are indicated before the membranes are removed. The membranes are then ished in TTBS, and incubated in TTBS containing 20% FCS and the polyclonal kappa casein antibodies diluted 1:100 for 2 h at room temperature. The membranes are ished two times for 5 minutes in TTBS at room temperature. Biotinylated goat-antirabbit IgG in TBS is added and the membranes incubated for 1 h at room temperature. The membranes are then ished again with TTBS two times for 5 min at room temperature. The conjugate of streptavidin and biotinylated alkaline phosphatase in TTBS is added followed by an incubation for 1 h at room temperature. The next step is to wash the membranes four times in TTBS for 5 min and then to rinse them three times in a buffer containing 50 mM Tris-HCl pH 9.8, 3 mM MgCl.sub.2, 50 μg/ml XP (5-bromo-4-chloro-3-indolylphosphate (Na-salt) and 100 μg/ml NBT (Nitroblue tetrasolium grade III).

The isolated plaques are purified by dilution and repeated screening. Phage DNA is prepared according to Sambrook et al. 1989 and the DNA preparations are digested with EcoRI. The digested DNA is separated by agarose electrophoresis and a number of EcoRI fragments are cloned into EcoRI digested and alkaline phosphatase treated pUC 18 plasmids and subsequently transformed into E. coli TG2. Transformants are selected on plates containing 50 μg/ml of carbenicillin, 40 μg/ml of X-gal (5-bromo-4-chloro-3-indolyl-.beta.-D-galactoside) and 1 mM IPTG (Isopropyl-.beta.-D-thiogalactoside). Plasmid DNA is analyzed from the transformants.

The nucleotide sequence encoding (SEQ ID NO:4, 5, 7, 8, 10, 11, 13, and 14) contained an open reading frame sufficient to encode the entire amino acid sequence of an altered kosher kappa casein protein.

### Example 3

### Assessing Reactivity of Mutated Bovine K-Casein

### The Sequences

The following optimized Bovine κ-casein sequences were cloned into pYes2, including 5′ BamH1 restriction site followed by AAAA Kozak sequence and 3′EcoR1 site.

### A=Wildtype (WT)

(SEQ ID No: 15)

GGTACCGGATCCAAAAATGATGAAGTCTTTCTTCTTGGTTGTTACTATTT
TGGCTTTGACTTTGCCATTTTTGGGTGCTCAAGAACAAAATCAAGAACAA
CCTATTAGATGTGAAAAGGACGAAAGATTCTTCTCAGATAAGATTGCTAA
GTACATTCCAATTCAATACGTTTTGTCTAGATATCCATCTTATGGTTTGA
ATTACTACCAACAAAAACCAGTTGCTTTGATTAACAATCAATTCTTGCCA
TATCCATATTATGCTAAACCAGCTGCTGTTAGATCTCCAGCTCAAATTTT
ACAATGGCAAGTTTTGTCTAATACTGTTCCAGCTAAATCTTGTCAAGCTC
AACCTACTACTATGGCTAGACATCCACATCCACATTTGTCTTTTATGGCT
ATTCCACCAAAAAAAAATCAAGATAAGACTGAAATTCCAACTATTAACAC
TATTGCTTCAGGTGAACCTACTTCTACTCCAACTACTGAAGCTGTTGAAT
CTACTGTTGCTACTTTGGAAGATTCTCCAGAAGTTATTGAATCTCCACCA
GAAATCAATACTGTTCAAGTTACTTCTACTGCTGTTCATCATCACCATCA
TCACTAATAAGAATTCGAGCTC.

B=Bovine κ-Casein Positive Control Mutation. Isoleucine at Position 94 Converted to Threonine (Ile_{—}94_Thr)

(SEQ ID No: 16)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATGTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAACTTTACAATG
GCAAGTTTTGTCTAATACTGTTCCAGCTAAATCTTGTCAAGCTCAACCTA
CTACTATGGCTAGACATCCACATCCACATTTGTCTTTTATGGCTATTCCA
CCAAAAAAAAATCAAGATAAGACTGAAATTCCAACTATTAACACTATTGC
TTCAGGTGAACCTACTTCTACTCCAACTACTGAAGCTGTTGAATCTACTG
TTGCTACTTTGGAAGATTCTCCAGAAGTTATTGAATCTCCACCAGAAATC
AATACTGTTCAAGTTACTTCTACTGCTGTTCATCATCACCATCATCACTA
ATAAGAATTC.

### C=Cysteine at Position #32 Changed to Serine (Cys 32 Ser)

(SEQ ID No: 17)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATCTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAATTTTACAATG
GCAAGTTTTGTCTAATACTGTTCCAGCTAAATCTTGTCAAGCTCAACCTA
CTACTATGGCTAGACATCCACATCCACATTTGTCTTTTATGGCTATTCCA
CCAAAAAAAAATCAAGATAAGACTGAAATTCCAACTATTAACACTATTGC
TTCAGGTGAACCTACTTCTACTCCAACTACTGAAGCTGTTGAATCTACTG
TTGCTACTTTGGAAGATTCTCCAGAAGTTATTGAATCTCCACCAGAAATC
AATACTGTTCAAGTTACTTCTACTGCTGTTCATCATCACCATCATCACTA
ATAAGAATTC.

### D=Amino Acids 104-111 Deleted (Del VPAKSCQA)

(SEQ ID No: 18)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATGTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAATTTTACAATG
GCAAGTTTTGTCTAATACTCAACCTACTACTATGGCTAGACATCCACATC
CACATTTGTCTTTTATGGCTATTCCACCAAAAAAAAATCAAGATAAGACT
GAAATTCCAACTATTAACACTATTGCTTCAGGTGAACCTACTTCTACTCC
AACTACTGAAGCTGTTGAATCTACTGTTGCTACTTTGGAAGATTCTCCAG
AAGTTATTGAATCTCCACCAGAAATCAATACTGTTCAAGTTACTTCTACT
GCTGTTCATCATCACCATCATCACTAATAAGAATTC.

### E=Amino Acids 104-111 Converted to Serines (VPAKSCQA SSSSSSSS)

(SEQ ID No: 19)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATGTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAATTTTACAATG
GCAAGTTTTGTCTAATACTTCTTCTTCATCTTCATCATCTTCTCAACCTA
CTACTATGGCTAGACATCCACATCCACATTTGTCTTTTATGGCTATTCCA
CCAAAAAAAAATCAAGATAAGACTGAAATTCCAACTATTAACACTATTGC
TTCAGGTGAACCTACTTCTACTCCAACTACTGAAGCTGTTGAATCTACTG
TTGCTACTTTGGAAGATTCTCCAGAAGTTATTGAATCTCCACCAGAAATC
AATACTGTTCAAGTTACTTCTACTGCTGTTCATCATCACCATCATCACTA
ATAAGAATTC.

### F=Cysteine at Position #109 Changed to Serine (Cys_{—}109_Ser)

(SEQ ID No: 20)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATGTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAATTTTACAATG
GCAAGTTTTGTCTAATACTGTTCCAGCTAAATCTTCTCAAGCTCAACCTA
CTACTATGGCTAGACATCCACATCCACATTTGTCTTTTATGGCTATTCCA
CCAAAAAAAAATCAAGATAAGACTGAAATTCCAACTATTAACACTATTGC
TTCAGGTGAACCTACTTCTACTCCAACTACTGAAGCTGTTGAATCTACTG
TTGCTACTTTGGAAGATTCTCCAGAAGTTATTGAATCTCCACCAGAAATC
AATACTGTTCAAGTTACTTCTACTGCTGTTCATCATCACCATCATCACTA
ATAAGAATTC.

### G=Cysteines at Positions #32 AND #39 Changed to Serines (Cys 32 109 Ser)

(SEQ ID No: 21)

GGATCCAAAAATGATGAAGTCTTTCTTCTTGGTTGTTACTATTTTGGCTT
TGACTTTGCCATTTTTGGGTGCTCAAGAACAAAATCAAGAACAACCTATT
AGATCTGAAAAGGACGAAAGATTCTTCTCAGATAAGATTGCTAAGTACAT
TCCAATTCAATACGTTTTGTCTAGATATCCATCTTATGGTTTGAATTACT
ACCAACAAAAACCAGTTGCTTTGATTAACAATCAATTCTTGCCATATCCA
TATTATGCTAAACCAGCTGCTGTTAGATCTCCAGCTCAAATTTTACAATG
GCAAGTTTTGTCTAATACTGTTCCAGCTAAATCTTCTCAAGCTCAACCTA
CTACTATGGCTAGACATCCACATCCACATTTGTCTTTTATGGCTATTCCA
CCAAAAAAAAATCAAGATAAGACTGAAATTCCAACTATTAACACTATTGC
TTCAGGTGAACCTACTTCTACTCCAACTACTGAAGCTGTTGAATCTACTG
TTGCTACTTTGGAAGATTCTCCAGAAGTTATTGAATCTCCACCAGAAATC
AATACTGTTCAAGTTACTTCTACTGCTGTTCATCATCACCATCATCACTA
ATAAGAATTC.

### DNA Synthesis and Cloning of Bovine κ-Casein and its Mutated Forms

DNA sequences were modified to optimize efficiency of translation (GeneArt AG, Regensburg, Germany). DNA was then chemically synthesized into cloned into pMA (GeneArt AG, Regensburg, Germany). A His₆ tag was placed at the 3′-terminus of the protein followed by two stop codons. The sequences of the native κ-casein and the various mutated κ-caseins generated are presented in sequences ID Nos: 15-21. Other optimization protocols resulting in different DNA sequences encoding the wild-type and mutated κ-casein can be used.

These 7 sequences encoded the following: A=Native κ-casein; B=Bovine κ-casein positive control, Isoleucine at position 94 converted to threonine (Ile_{—}94_Thr); C=Cysteine at position #32 changed to serine (Cys_{—}32_ser); D=Amino Acids 104-111 deleted (Del_VPAKSCQA); E=Amino Acids 104-111 converted to serines (VPAKSCQA_SSSSSSSS); F=Cysteine at position #109 changed to serine (Cys_{—}109_Ser); G=Cysteines at positions #32 AND #39 changed to serines (Cys_{—}32_{—}109_Ser). In other experiments other single or combined mutations were generated.

In other experiments the His₆ tag was added during the two-step PCR procedure (Qiagen, Hilden Germany, see below). In addition a BamH1 and EcoR1 sites were added at the 3′ and 5″ end, respectively for ease of subsequent cloning steps. Native κ-casein and mutated forms were subcloned into pYes2 vector (Invitrogen Life Technologies). In other embodiments any other cloning vectors can be utilized. The encoded DNA was then used as substrate for a first step PCR reaction with the goal to generate a template suitable for in vitro transcription reaction (see herein below).

### Expression of Bovine Kappa-Casein and Mutated Forms In Vitro

An in insect cell based expression system (Qiagen) was utilized to generate recombinant bovine κ-casein and its mutated forms in vitro. A two-step PCR process was used to generate linear DNA that served as templates for the in vitro translation step. During the first PCR step, gene-specific primers with 5′ tails are used. The gene-specific primers used to generate the first-round PCR product are listed in Table 4. In the second step, the 5′ tails serve as hybridization sites for primers (provided by the manufacturer) used in a second round PCR (EasyXpress Linear Template kit, Qiagen). In addition, a T7 promoter, regulatory elements, 5′ and 3′untranslated regions are added in the second-step to facilitate the expression of the cloned DNA. His₆ tags or other known tags from the state of the art can be added for ease of detection and purification (EasyXpress Linear Template kit, Qiagen). After the second-step PCR, the PCR-product was used as a template in an insect cell in vitro transcription/translation system, as per the manufacturers' directions (EasyXpress Insect Kit II, Qiagen). Other transcription/translation systems can also be utilized.

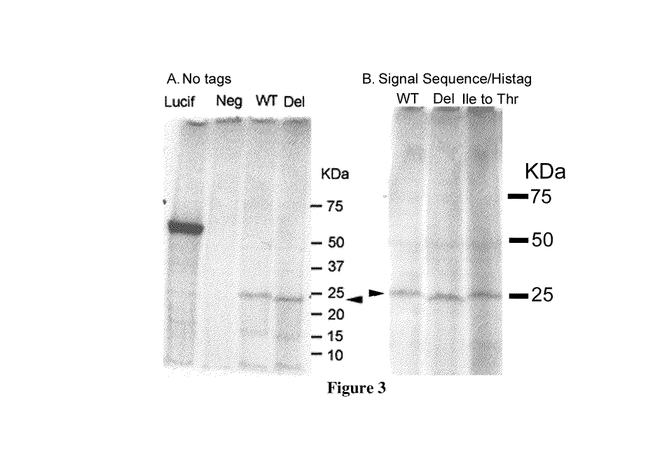

Briefly, 500 ng of PCR product template was added for each 25 microliter reaction containing transcription buffer, nucleotriphosphate and enzyme mix and incubated for two hours at 37 degrees Celsius, to generate capped mRNA. This mRNA is cleaned up using a DyeEx gel-filtration spin column before its addition to the translation reaction. The translation reaction containing insect-cell reaction buffer, insect cell extract, template mRNA, and insect cell “energy mix” was incubated for 90 minutes at 27 degrees Celsius while spinning at 220 rpm in an orbital shaker. The reaction mixture is frozen at −70 degrees Celsius until further use in skin-prick testing (see below), purification or analysis by SDS-PAGE. Analysis by SDS-PAGE can detect the generated κ-casein either by immunoblot with anti-bovine casein antibodies or anti-His₆ antibodies, or by the addition of ³⁵S-methionine and ³⁵S-cysteine mixtures that gets incorporated into the protein during the reaction. One such example of the latter is illustrated in the FIG. 3. Presented are two autoradiograms in which the native and mutated κ-casein are expressed in insect cell extracts either without the addition of tags or with the addition of the signal sequence on the 5′ end and a His₆ at the 3′ end.

Purification procedures can use the employed tags in immunoaffinity and/or chromatographic methods as known in the art. In addition, chromatographic procedures for the purification of kappa-casein are well known to the skilled artisian.

TABLE 4

Gene Specific first-round primer pairs

| | |
|---|---|
| Forward Primer | Reverse Primer |

| | | |
|---|---|---|
| No tag | Agaaggagataaacaatgcaag | Cttggttagttagttattaaaca |
| | Aacaaaatcaagaacaa | Gcagtagaagtaacttg |
| | SEQ ID NO: 22 | SEQ ID NO: 23 |
| No tag with signal sequence | Agaaggagataaacaatgatgaa | Cttggttagttagttattaaacag |
| | Gtctttcttcttggtt | Cagtagaagtaacttg |
| | SEQ ID NO: 24 | SEQ ID NO: 25 |
| C-terminus | Agaaggagataaacaatgcaaga | Tggtgatggtggtgaccccaaa |
| His₆ tag | Acaaaatcaagaacaa | Cagcagtagaagtaacttg |
| | SEQ ID NO: 26 | SEQ ID NO: 27 |
| C-terminus | Agaaggagataaacaatga | Tggtgatggtggtgaccccaaa |
| His₆ tag with signal sequence | Tgaagtctttcttcttggtt | Cagcagtagaagtaacttg |
| | SEQ ID NO: 28 | SEQ ID NO: 29 |
| C-terminus using His₆ tag added to | Agaaggagataaacaatgca | Cttggttagttagttattattagtgat |
| k-casein sequence in plasmid | Agaacaaaatcaagaacaa | Gatggtgatgatg |
| | SEQ ID NO: 30 | SEQ ID NO: 31 |

The expressed proteins were then assessed for reactivity in previously identified IgE-mediated cow's milk protein patients. We used the bovine κ-casein sequence in which the isoleucine at position #94 was converted to a serine (sequence 16 (B)) as a positive control for these experiments. This conversion should unlikely diminish its allergenicity since it is encoded in other kosher animals (for example, goat). As demonstrated in the table below, mutated κ-casein sequence “E” was negative in the two patients studied and mutation “D” in ⅔ patients. Of note, the patient who responded to the deleted form (patient #3, “D”) was strongly reactive to nonkosher animal's milk. He still had a negative reaction in mutation “F”. All were positive to the Bovine κ-casein control mutation (“B”)

TABLE 5
Skin Prick Test Results of insect cell extracts

| | | | Mutated | | |
|---|---|---|---|---|---|
| | | Mutated | κ-casein | Mutated | |
| | Bovine κ-casein | κ-casein | (AA 104-111, | κ-casein | Mutated κ-casein |
| | (control | AA 104-111, | VPAKSCQA | Cysteine-32 | Cysteine-109 |
| | mutation | VPAKSCQA | converted to) | converted to | converted to |
| Patient | Ile94 to Ser) | Deleted. | to eight serines | to Serine | to Serine |
| # | Sequence “B” | Sequence “D” | Sequence “E” | Sequence “C” | Sequence “F” |
| 1. | + | − | − | n.d. | n.d |
| 2. | + | − | − | n.d. | n.d |
| 3*. | + | + | n.d. | + | − |

*This patient was also strongly reactive to nonkosher animal's milk. A reaction was considered positive (+ in table) if the size of the wheal was equal or greater than the Histamine (1 mg/ml) control reaction. n.d. = no data, Insect cell extract alone gave no reaction (Data not shown).

### Example 4

### Expression of Altered Kosher Kappa Casein in Bacterial Systems

In order to produce recombinant altered kosher kappa casein in E. coli, the altered kosher kappa casein encoding sequence is introduced into two different vectors. One vector contains a signal sequence in front of the altered kosher kappa casein encoding sequence whereas the other lacks such a signal sequence.

### Example 5

### Expression of Altered Kosher Kappa Casein in Mammalian Cells

To produce altered kosher kappa casein in mammalian cell culture systems, the altered kosher kappa casein cDNA is introduced into an eukaryotic expression vector.

### Example 6

### In Vitro Maturation, Fertilization and Culture of Bovine Oocytes

Immature oocytes are obtained in large quantity (400-600/day) by aspirating follicles of ovaries obtained at abattoirs. Immature oocytes are cultures for a period in vitro before they are competent to be fertilized. Once “matured”, oocytes are fertilized with sperm which has also been matured, or “capacitated” in vitro. The pronuclei of the fertilized oocyte is then injected with the transgene coding for the expression and secretion of altered kosher kappa casein Zygotes resulting from this in vitro fertilization and microinjection are then cultured to the late morula or blastocyst stage (5-6 days) in medium prepared, or “conditioned” by oviductal tissue. Blastocysts are then transferred non-surgically to recipient bovine species for the balance of gestation or analyzed for integration of the transgene as described herein.

### In Vitro Maturation (IVM)

Ovaries are obtained immediately after slaughter at local abattoirs and oocytes are recovered. Alternatively, oocytes are obtained from living bovine species by surgical, endoscopic, or transvaginal ultrasonic approaches. In all cases, oocytes are aspirated from ovarian follicles (2-10 mm diameter). After washing, oocytes are placed in a maturation medium such as a medium consisting of M199 supplemented with 10% fetal calf serum, and incubated for 24 hours at 39° C.

### In Vitro Fertilization (IVF)

Matured oocytes are fertilized with either fresh or thawed sperm. Sperm is prepared for fertilization by first obtaining a population of sperm enriched for motility by a “swim-up” separation technique (Parrish et al. (1986) Theriogenology 25, 591-600). Motile sperm is then added to a fertilization medium, consisting of a modified Tyrode's solution (Parrish et al. (1986) supra) supplemented with heparin to induce sperm capacitation (Parrish et al. (1988) Biol. Reprod. 38, 1171-1180). Capacitation constitutes the final sperm maturation process which is essential for fertilization. Sperm and oocytes are co-cultured for 18 hours. A useful feature of this IVF method is that (in the case of frozen sperm) consistent, repeatable results are obtained once optimal fertilization conditions for a particular ejaculate have been defined (Parrish et al. (1986) supra).

### Example 7

### Microinjection of Altered Kosher Kappa Casein Transgene Into Bovine Pronuclei

The DNA fragment containing the altered kosher kappa casein expression system is excised from the vector by digestion with the appropriate restriction enzyme(s) and separated on agarose gels. The fragment is purified by electroelution, phenol and chloroform extraction and ethanol precipitation (Maniatis et al.). The DNA fragment is dissolved in and dialyzed in 10 mM tris, 0.1 mM EDTA pH 7.2 at a concentration of 1 to 2 μg/ml. Microinjection needles are filled with the dialyzed DNA solution.

Before in vitro fertilization, cumulus cells are removed from the egg by either vortexing at maximum speed for 2 minutes or pipetting the eggs up and down several times in a standard micropipette. Bovine pronuclei are injected with an additional centrifugation step in order to visualize the pronuclei. The injection takes place 18-24 hours after fertilization. The time varies depending on the bull used as a source of semen. Different batches of semen cause the nuclei to become visible at different times.

Bovine oocytes, matured and fertilized in vitro, are spun in an eppendorf tube in 1 ml of tyrodes-hepes solution at 14500 g for eight minutes Biol. Reprod. 32, 645-651). The embryos are transferred to a drop of tyrodes-hepes solution on a microscope slide covered with paraffin oil. Using a hydraulic system the oocytes are fixed to the egg holder in such a way that both the pronuclei are visible (using interference-contrast or phase contrast optics). If necessary, the oocytes are rolled to change their position on the egg holder to visualize the pronuclei. The injection needle is brought into the same sharp focus of one of the pronuclei. The needle is then advanced through the zona pellucida, cytoplasm into the pronucleus. A small volume of 13 pl is injected (containing 20-100 DNA copies) into the pronucleus either by using a constant flow or a pulse flow (using a switch) of DNA solution out of the needle. Alternatively, two cell stage embryos are spun as described and the nuclei of both blastomers are injected as described. The injected embryos are then transferred to a drop of co-culture medium as described in Example 6 in order to develop to the morula or blastocyst stage.

### Example 8

### Early Detection of Transgenesis with Altered Kosher Kappa Casein Transgene

Upon the microinjection of a construct, the oocyte is cultured. A proper site of each embryo is cleaved and subjected to lysis, proteolysis, amplifications, and digestion.

### Example 9

### Production of Altered Kosher Kappa Casein in Milk of Bovine Species

Bovine morula developed from microinjected oocytes are split according to the method of Donahue (Donahue, S. (1986) Genetic Engineering of Animals, ed. J. Warren Evans et al., Plenum). One half of the morula is kept in culture to develop into blastocysts. The other half is subjected to DNA analysis. When the result of this analysis is known, the morula kept in culture are developed into a blastocyst or as a source for nuclear transfer into enucleated zygotes. Blastocyst transfer into synchronized cows is performed according to the method of Betteridge (Betteridge, K. J. (1977) in: Embryo transfer in farm animals: a review of techniques and applications).

What is claimed is:

1. An isolated kosher casein polypeptide, comprising: (1) at least one altered amino acid in the amino acid stretch encoded by SEQ ID No: 1 or SEQ ID No: 2 of a kosher casein polypeptide; or (2) a substitution or a deletion in the position of Cys 31, Cys 32, or Cys 31 and Cys 32 of a kosher casein polypeptide.

2. The isolated polypeptide of claim 1, lacking the 8 amino acids encoded by SEQ ID No: 1 or SEQ ID No: 2.

3. The isolated polypeptide of claim 1, wherein said kosher animal is a bovine, a deer, a giraffe, a cow, a buffalo, a goat, a sheep or a pronghorn.

4. The isolated polypeptide of claim 1, wherein said polypeptide is produced in a eukaryotic cell or a eukaryotic cell extract.

5. The isolated polypeptide of claim 1, wherein said polypeptide is produced in a kosher animal.

6. The isolated polypeptide of claim 1, comprising an amino acid sequence selected from sequences set forth in SEQ ID NO: 4, 5, 7, 8, 10, 11, 13, or 14.

7. The isolated polypeptide of claim 1, comprising: (1) at least one altered amino acid in the amino acid stretch encoded by SEQ ID No: 1 or SEQ ID No: 2; and (2) a substitution or a deletion in the position of Cys 31, Cys 32, or Cys 31 and Cys 32.

8. A DNA molecule encoding the polypeptide of claim 1.

9. A composition comprising the polypeptide of claim 1.

10. The composition of claim 9, wherein said polypeptide is lacking the 8 amino acids encoded by SEQ ID No: 1 or SEQ ID No: 2.

11. The composition of claim 9, wherein said composition is a food additive or milk.

12. The composition of claim 9, wherein said polypeptide comprises an amino acid sequence selected from sequences set forth in SEQ ID NO: 4, 5, 7, 8, 10, 11, 13, or 14.

13. A method of reducing the allergenicity of a kosher casein polypeptide, comprising the step of (1) altering at least one amino acid of said polypeptide in the amino acid stretch encoded by SEQ ID No: 1 or SEQ ID No: 2 of a kosher casein polypeptide; or (2) substituting or a deleting Cys 31, Cys 32, or both Cys 31 and Cys 32 of a kosher casein polypeptide, thereby reducing the allergenicity of a casein polypeptide.

14. The method of claim 13, wherein said polypeptide comprises an amino acid sequence selected from sequences set forth in SEQ ID NO: 4, 5, 7, 8, 10, 11, 13, or 14.

15. The method of claim 13, wherein said reducing allergenicity to a casein polypeptide comprises deleting the 8 amino acids encoded by SEQ ID No: 1 or SEQ ID No: 2.

16. The method of claim 13, wherein said reducing allergenicity to a casein polypeptide comprises substituting or deleting Cys 31, Cys 32, or both Cys 31 and Cys 32.

17. The method of claim 13, wherein said kosher animal is a bovine, a deer, a giraffe, a cow, a buffalo, a goat, a sheep or a pronghorn.

18. The method of claim 13, wherein said polypeptide is produced in a eukaryotic cell or a eukaryotic cell extract.

19. The method of claim 13, wherein said polypeptide is produced in a kosher animal.

20. An expression system comprising a first DNA sequence encoding a kosher animal casein polypeptide, said kosher animal casein polypeptide comprises: (1) at least one altered amino acid in the amino acid stretch encoded by SEQ ID No: 1 or SEQ ID No: 2 of a kosher casein polypeptide; or (2) a substitution or a deletion in the position of Cys 31, Cys 32, or Cys 31 and Cys 32 of a kosher casein polypeptide.

21. A replicable expression vector which comprises the expression system of claim 20.

22. The expression system of claim 20, further comprising a second DNA sequence preceding said first sequence and operably linked in phase thereto, said second DNA sequence encoding a secretory signal polypeptide functional in mammalian cells.

23. The expression system of claim 20, wherein said casein is kappa casein.

24. The expression system of claim 20, wherein said kosher animal casein polypeptide comprising at least one altered amino acid in the amino acid stretch encoded by SEQ ID No: 1 or SEQ ID No: 2 is a polypeptide comprising an amino acid sequence as set fourth in SEQ ID No: 4, 5, 7, 8, 10, 11, 13, or 14.

25. A transgenic kosher mammal, comprising a transgene encoding a kosher casein polypeptide, comprising: (1) at least one altered amino acid in the amino acid stretch encoded by SEQ ID No: 1 or SEQ ID No: 2 of a kosher casein polypeptide; or (2) a substitution or a deletion in the position of Cys 31, Cys 32, or Cys 31 and Cys 32 of a kosher casein polypeptide.

26. A method for inducing desensitization or oral tolerance in a subject, comprising administering to said subject a composition comprising an effective amount of the polypeptide of claim 1, thereby inducing desensitization or oral tolerance in a subject.

27. A method of diagnosing a subject destined to remain allergic to a kappa-casein polypeptide, comprising the step of conducting a Skin Prick comprising at least one polypeptide of claim 1, thereby diagnosing a subject destined to remain allergic to a kappa-casein polypeptide.


SEQUENCE LISTING (31 sequences)

SEQ ID NO: 1 - protein, 8 aa, Artificial Sequence
  8 amino acids segment derived from Kosher Casein protein
VPAKSCQD

SEQ ID NO: 2 - protein, 8 aa, Artificial Sequence
  8 amino acids segment derived from Kosher Casein protein
VPAKSCQA

SEQ ID NO: 3 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 4 - protein, 182 aa, Artificial Sequence
  Mutated kosher casein kappa protein
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTQPTTMARHPHPHLSFMAIPPKKNQDKTEIPTINTIASGEPTSTPTTEAVESTVATLEDSPEVIESPPEINTVQVTSTAV

SEQ ID NO: 5 - protein, 183 aa, Artificial Sequence
  Mutated kosher casein kappa protein
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTAQPTTMARHPHPHLSFMAIPPKKNQDKTEIPTINTIASGEPTSTPTTEAVESTVATLEDSPEVIESPPEINTVQVTSTAV

SEQ ID NO: 6 - protein, 188 aa, Bubalus bubalis
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPITEAIENTVATLEASSEVIESVPETNTAQVTST

SEQ ID NO: 7 - protein, 180 aa, Artificial Sequence
  Mutated kosher casein kappa protein
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTQPTTMTRHPHPHLSFMAIPPKKNQDKTEIPTINTIVSVEPTSTPITEAIENTVATLEASSEVIESVPETNTAQVTST

SEQ ID NO: 8 - protein, 181 aa, Artificial Sequence
  Mutated kosher casein kappa protein
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTAQPTTMTRHPHPHLSFMAIPPKKNQDKTEIPTINTIVSVEPTSTPITEAIENTVATLEASSEVIESVPETNTAQVTST

SEQ ID NO: 9 - protein, 192 aa, Capra hircus
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 10 - protein, 184 aa, Artificial Sequence
  Mutated kosher casein kappa protein
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTQPTTLARHPHPHLSFMAIPPKKDQDKTEVPAINTIASAEPTVHSTPTTEAIVNTVDNPEASSESIASASETNTAQVTSTEV

SEQ ID NO: 11 - protein, 185 aa, Artificial Sequence
  Mutated kosher casein kappa protein
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTDQPTTLARHPHPHLSFMAIPPKKDQDKTEVPAINTIASAEPTVHSTPTTEAIVNTVDNPEASSESIASASETNTAQVTSTEV

SEQ ID NO: 12 - protein, 192 aa, Ovis aries
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 13 - protein, 184 aa, Artificial Sequence
  Mutated kosher casein kappa protein
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAQPTAMARHPHPHLSFMAIPPKKDQDKTEIPAINTIASAEPTVHSTPTTEAVVNAVDNPEASSESIASAPETNTAQVTSTEV

SEQ ID NO: 14 - protein, 185 aa, Artificial Sequence
  Mutated kosher casein kappa protein
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNADQPTAMARHPHPHLSFMAIPPKKDQDKTEIPAINTIASAEPTVHSTPTTEAVVNAVDNPEASSESIASAPETNTAQVTSTEV

SEQ ID NO: 15 - protein, 622 aa, Bos taurus
GGTACCGGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATGTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTGTTCCAGCTAAATCTTGTCAAGCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTCGAGCTC

SEQ ID NO: 16 - protein, 610 aa, Artificial Sequence
  Isoleucine at position 94 converted to threonine (Ile_94_Thr)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATGTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAACTTTACAATGGCAAGTTTTGTCTAATACTGTTCCAGCTAAATCTTGTCAAGCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 17 - protein, 610 aa, Artificial Sequence
  C= Cysteine at position #32 changed to serine (Cys_32_ser)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATCTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTGTTCCAGCTAAATCTTGTCAAGCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 18 - protein, 586 aa, Artificial Sequence
  D=Amino Acids 104-111 deleted (Del_VPAKSCQA)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATGTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 19 - protein, 610 aa, Artificial Sequence
  E=Amino Acids 104-111 converted to serines (VPAKSCQA_SSSSSSSS)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATGTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTTCTTCTTCATCTTCATCATCTTCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 20 - protein, 610 aa, Artificial Sequence
  F= Cysteine at position #109 changed to serine (Cys_109_Ser)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATGTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTGTTCCAGCTAAATCTTCTCAAGCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 21 - protein, 610 aa, Artificial Sequence
  G=Cysteines at positions #32 AND #39 changed to serines (Cys_32_109_Ser)
GGATCCAAAAATGATGAAGTCTTTCTTCTTGGTTGTTACTATTTTGGCTTTGACTTTGCCATTTTTGGGTGCTCAAGAACAAAATCAAGAACAACCTATTAGATCTGAAAAGGACGAAAGATTCTTCTCAGATAAGATTGCTAAGTACATTCCAATTCAATACGTTTTGTCTAGATATCCATCTTATGGTTTGAATTACTACCAACAAAAACCAGTTGCTTTGATTAACAATCAATTCTTGCCATATCCATATTATGCTAAACCAGCTGCTGTTAGATCTCCAGCTCAAATTTTACAATGGCAAGTTTTGTCTAATACTGTTCCAGCTAAATCTTCTCAAGCTCAACCTACTACTATGGCTAGACATCCACATCCACATTTGTCTTTTATGGCTATTCCACCAAAAAAAAATCAAGATAAGACTGAAATTCCAACTATTAACACTATTGCTTCAGGTGAACCTACTTCTACTCCAACTACTGAAGCTGTTGAATCTACTGTTGCTACTTTGGAAGATTCTCCAGAAGTTATTGAATCTCCACCAGAAATCAATACTGTTCAAGTTACTTCTACTGCTGTTCATCATCACCATCATCACTAATAAGAATTC

SEQ ID NO: 22 - DNA, 39 bp, Artificial Sequence
  FWD Primer-no tag
agaaggagataaacaatgcaagaacaaaatcaagaacaa

SEQ ID NO: 23 - DNA, 40 bp, Artificial Sequence
  Reverse primer-no tag
cttggttagttagttattaaacagcagtagaagtaacttg

SEQ ID NO: 24 - DNA, 39 bp, Artificial Sequence
  FWD Primer-No tag with signal sequence
agaaggagataaacaatgatgaagtctttcttcttggtt

SEQ ID NO: 25 - DNA, 40 bp, Artificial Sequence; identical to SEQ ID NO: 23
  Reverse Primer-No tag with signal sequence

SEQ ID NO: 26 - DNA, 39 bp, Artificial Sequence; identical to SEQ ID NO: 22
  FWD Primer-C-terminus HIS6 Tag

SEQ ID NO: 27 - DNA, 41 bp, Artificial Sequence
  Reverse Primer-C-terminus His6 Tag
tggtgatggtggtgaccccaaacagcagtagaagtaacttg

SEQ ID NO: 28 - DNA, 39 bp, Artificial Sequence; identical to SEQ ID NO: 24
  FWD Primer-C-terminus His6 tag with signal sequence

SEQ ID NO: 29 - DNA, 41 bp, Artificial Sequence; identical to SEQ ID NO: 27
  Reverse Primer-C-terminus His6 tag with signal sequence

SEQ ID NO: 30 - DNA, 39 bp, Artificial Sequence; identical to SEQ ID NO: 22
  FWD Primer-C-terminus using His6 tag added to k-casein sequence in plasmid

SEQ ID NO: 31 - DNA, 40 bp, Artificial Sequence
  Reverse Primer-C-terminus using His6 tag added to k-casein sequence in plasmid
cttggttagttagttattattagtgatgatggtgatgatg